(19) United States
(12) Patent Application Publication
(10) Pub. No.: US 2025/0331992 A1
(43) Pub. Date: Oct. 30, 2025

(54) Device For Mechanical Securement To Hard Tissue

(71) Applicant: The Trustees of Columbia University in the City of New York, New York, NY (US)

(72) Inventors: Alexander Kell, Jamaica Plain, NY (US); Elias Issa, New York, NY (US)

(21) Appl. No.: 19/039,244

(22) Filed: Jan. 28, 2025

Related U.S. Application Data
(63) Continuation-in-part of application No. PCT/US2023/071262, filed on Jul. 28, 2023.
(60) Provisional application No. 63/392,918, filed on Jul. 28, 2022.
(60) Provisional application No. 63/392,921, filed on Jul. 28, 2022.

(51) Int. Cl. A61F 2/36; A61F 2/30

(52) U.S. Cl. CPC A61F 2/3601; A61F 2/30728; A61F 2/30744; A61F 2/30749

(57) ABSTRACT

A device for mechanical securement to hard tissue of a subject, the hard tissue having an end surface defining a direction of curvature such that over at least a portion of the surface, a total curvature of at least 180 degrees is defined, the device comprising a body portion comprising at least four contact points, three contact points of the four contact point span over 180 degrees of total curvature of the end surface in a plane defined by the three contact points, and a fourth contact point outside the plane, the four contact points providing mechanical securement of the body portion to the hard tissue.

### CROSS-REFERENCE TO RELATED APPLICATIONS

This application is a continuation-in-part of PCT/US2023/071262, filed Jul. 28, 2023, which claims the benefit of priority to U.S. Provisional Application No. 63/392,918, filed Jul. 28, 2022, and U.S. Provisional Application No. 63/392,921, filed Jul. 28, 2022, the entire contents of each of which are incorporated herein by reference for any and all purposes.

### STATEMENT OF GOVERNMENT SUPPORT

This invention was made with government support under grants DC017628, NS116739, and EY022671 awarded by the National Institutes of Health. The government has certain rights in the invention.

### FIELD

The present disclosure generally relates to medical devices, implants, prosthetics or appliances for securement to a subject for diagnostic and/or therapeutic purposes, and more particularly to devices for mechanical securement to hard tissue.

### BACKGROUND

Securement to hard tissue in the body is found in a number of applications. Generally, hard tissue is understood to refer to bones and teeth. Such securement includes, for example, prosthetics to replace damaged or lost limbs, implanting devices, and dental implants on teeth. Mechanically securing a device is often done using bone screws or other fasteners that penetrate the bone. Fastening can also be done using cements or glues, but these often are not as biocompatible, mechanically strong, and long-lasting as relying on more mechanical solutions.

An implant at a high-load location, such as the hip joint, needs to be able to withstand strong forces over decades of use. As a result of suboptimal mechanics, the current state-of-the-art in hip implants still experience failure both in the short-term (in the first year after the implant surgery) and in the long run after decades of load-bearing use. Hip implant failures not only decrease quality of life for the patient but also result in a risky, revision surgery where the femur and hip socket may be further compromised. Especially for the increasing cohort of younger patients who need longer implant life and for the increasingly long life expectancy and who tend to be more active, significant improvement in hip replacement outcomes would still be desired whether in better function, longevity, or both. The same is true for other anatomical locations at which joint deterioration can occur, such as the elbow, shoulder, or indeed any location where an implant may be needed because of injury, osteoarthritis, or for any other reason. For example, shoulder replacements where high mobility of the joint in conjunction with smaller bone mass for penetrating implants leads to an undesirable rate of failures.

What is needed is an improved bone implant design that overcome the disadvantages of conventional designs.

For cranial implants, infiltration of the skull entails exposing delicate soft tissue such as the brain and dura including vasculature. And for securing hardware otherwise to the skull, current devices such as deep brain stimulators are implanted elsewhere on the body which requires a separate surgical procedure and raises cosmetic issues given the often visible scar and bulge introduced.

For sensors and actuators interfacing the brain, particularly as we move towards a world of neural augmentation via elective, commercial implants, what is needed is a secure, cosmetically low-profile method of attachment to the skull with the opportunity for hair to conceal. If the device does not require penetrating skull this limits the risk of the surgical procedure and would increase appeal to a larger part of the population.

### SUMMARY

In one aspect of the subject matter, provided is prosthesis for securement to an end portion of a bone representing a first member of a joint in a subject, comprising a cap portion that is configured for fitting over the end portion of the bone; and, a collar comprising a first collar portion and a second collar portion, at least one of said first collar portion and said second collar portion being coupled to the cap portion, wherein an interior surface of the collar conforms to a portion of an outer surface of a side portion of the bone extending from the end portion, and wherein the first collar portion is configured to be coupled to the second collar portion to at least partially engage the outer surface of the side portion of the bone to provide mechanical securement of the collar with the bone.

In some aspects, provided is a hip joint prosthesis for securement to the neck of the femur of a subject following removal of the femoral head, including a substantially spherical ball portion for replacement of the femoral head; and a collar coupled to the ball portion and comprising a first collar portion and a second collar portion, wherein an interior surface of the collar conforms to a portion of the outer surface of the neck of the femur; wherein the first collar portion is configured to be coupled to the second collar portion to at least partially engage the outer surface of the neck of the femur to provide mechanical securement of the collar with the femur. In some embodiments, the neck of the femur has an end surface defining a direction of curvature such that over at least one such portion of the end surface, a total curvature of at least 180 degrees is defined, the collar includes at least four contact points, wherein three contact points of the four contact points span over 180 degrees of total curvature of the end surface in a plane defined by the three contact points, and a fourth contact point outside the plane, the four contact points providing mechanical securement of the body portion to the hard tissue.

In some embodiments, a bolt is provided to secure the first collar portion and the second collar portion. In some embodiments, the first collar portion is symmetric with the second collar portion. In some embodiments, the first collar portion and the second collar portion overlap the line of attachment of border of synovial membrane. In some embodiments, the first collar portion and the second collar portion extends to the line of reflection of the synovial membrane.

In some embodiments, the ball portion is integral with the first collar portion. In some embodiments, the ball portion comprises first and second substantially hemispherical components, and wherein the first substantially hemispherical component is integral with the first collar portion and the second substantially hemispherical component is integral with the second collar portion. In some embodiments, the ball portion is removably coupleable with the first collar portion and the second collar portion.

In another aspect of the subject matter, an implant for securement to the end surface tissue of a subject is provided, including a first arm; a second arm; and a third arm extending from one or both of the first arm and the second arm, the first arm, the second arm and the third arm comprising at least four contact points, wherein three contact points of the four contact points span over 180 degrees of total curvature of the end surface in a plane defined by the three contact points, and a fourth contact point outside the plane, the four contact points providing mechanical securement of the body portion to the hard tissue.

In some embodiments, the implant further includes a first body portion and a second body portion, and wherein the first arm extends from the first body portion and the second arm extends from the second body portion, and further comprising a securement mechanism for coupling the first and second body portions about the skull of the subject.

In some embodiments, the hard tissue is the skull of the subject. In some embodiments, the first contact point is configured for engagement with the left temporal pole of the skull of the subject. In some embodiments, the second contact point is configured for engagement with the right temporal pole of the skull of the subject. In some embodiments, the first contact point and second contact point are configured for engagement with the nuchal crests of the skull of the subject. In some embodiments, the fourth contact point is configured for engagement with the occipital pole protrusion of the skull of the subject.

In some embodiments, the implant is fabricated from titanium. In some embodiments, the subject is a marmoset monkey, macaque or mouse.

In some embodiments, the first arm and the second arm define an access window extending therethrough.

In another aspect of the disclosed subject matter, a device for mechanical securement to hard tissue of a subject is provided, the hard tissue having an end surface defining a direction of curvature such that over at least one such portion, a total curvature of at least 180 degrees is defined, the device including a body portion having at least four contact points, three contact points of the four contact point span over 180 degrees of total curvature of the end surface in a plane defined by the three contact points, and a fourth contact point outside the plane, the four contact points providing mechanical securement of the body portion to the hard tissue.

### BRIEF DESCRIPTION OF THE DRAWINGS

Various embodiments of the present disclosure can be further explained with reference to the attached drawings, wherein like structures are referred to by like numerals throughout the several views. The drawings shown are not necessarily to scale, with emphasis instead generally being placed upon illustrating the principles of the present disclosure. Therefore, specific structural and functional details disclosed herein are not to be interpreted as limiting, but merely as a representative basis for teaching one skilled in the art to variously employ one or more illustrative embodiments.

FIGS. 1-3 illustrate human hip anatomy and physiology.

FIG. 4A illustrates an exterior front view of an exemplary embodiment of the disclosed subject matter.

FIG. 4B illustrates an exterior side view of the exemplary embodiment of FIG. 4A.

FIG. 4C illustrates an interior view of the exemplary embodiment of FIG. 4A.

FIG. 4D illustrates an isometric view of the exemplary embodiment of FIG. 4A.

FIGS. 5A-5D illustrate stages in the installation of the implant of FIGS. 4A-4D.

FIG. 6 is a side view of an implant installed on the end of a bone in accordance with another exemplary embodiment of the disclosed subject matter.

FIG. 7A is a side view with parts separated of another exemplary embodiment of the disclosed subject matter.

FIG. 7B is an isometric view from above with parts separated of the device of FIG. 7A.

FIG. 7C is a front exterior view with parts separated of the device of FIG. 7A.

FIG. 7D is a front interior view with parts separated of the device of FIG. 7A.

FIGS. 8A-8D illustrate stages in the installation of the implant of FIGS. 7A-7D.

FIG. 9 is a side view of an implant installed on the end of a bone in accordance with a further exemplary embodiment of the disclosed subject matter.

FIGS. 10A-B are cross-sectional views of the device implanted on a bone of the patient illustrating mechanical forces.

FIGS. 11A-C schematically illustrate a technique for manufacturing and installing the implant.

FIG. 12 schematically illustrates a technique for manufacturing and installing the implant.

FIG. 13 illustrates the femur anatomy.

FIGS. 14 and 15A-B illustrate exemplary femur cuts to remove a portion of the ball joint.

FIG. 16A illustrates an interior view with parts separated of another exemplary embodiment of the disclosed subject matter.

FIG. 16B illustrates an exterior side view of the exemplary embodiment of FIG. 16A.

FIG. 16C illustrates an isometric view of the exemplary embodiment of FIG. 16A.

FIGS. 17A-B illustrate side views of the implant of FIGS. 16A-C installed on a hip bone in accordance with another exemplary embodiment of the disclosed subject matter.

FIG. 18A illustrates an interior view with parts separated of another exemplary embodiment of the disclosed subject matter.

FIG. 18B illustrates an exterior side view of the exemplary embodiment of FIG. 18A.

FIG. 18C illustrates an isometric view of the exemplary embodiment of FIG. 18A.

FIGS. 19A-B illustrate side views of the implant of FIGS. 18A-C installed on a=bone in accordance with another exemplary embodiment of the disclosed subject matter.

FIG. 20A illustrates an interior view with parts separated of a further exemplary embodiment of the disclosed subject matter.

FIG. 20B illustrates an exterior side view of the exemplary embodiment of FIG. 20A.

FIG. 20C illustrates an isometric view of the exemplary embodiment of FIG. 20A.

FIGS. 21A-B illustrate side views of the implant of FIGS. 20A-C installed on a bone in accordance with another exemplary embodiment of the disclosed subject matter.

FIG. 22A illustrates an interior view with parts separated of a further exemplary embodiment of the disclosed subject matter.

FIG. 22B illustrates an exterior side view of the exemplary embodiment of FIG. 22A.

FIG. 22C illustrates an isometric view of the exemplary embodiment of FIG. 22A.

FIGS. 23A-B illustrate side views of the implant of FIGS. 22A-C installed on a hip bone in accordance with another exemplary embodiment of the disclosed subject matter.

FIG. 24 illustrates an interior view with parts separated of a still further exemplary embodiment of the disclosed subject matter.

FIGS. 25A-C illustrate side views of the implant of FIG. 24 installed on a bone in accordance with another exemplary embodiment of the disclosed subject matter.

FIG. 26 illustrates an interior view with parts separated of yet another exemplary embodiment of the disclosed subject matter.

FIGS. 27A-C illustrate side views of the implant of FIG. 26 installed on a bone in accordance with another exemplary embodiment of the disclosed subject matter.

FIG. 28 illustrates an interior view with parts separated of a further exemplary embodiment of the disclosed subject matter.

FIGS. 29A-C illustrate side views of the implant of FIG. 28 installed on a bone in accordance with another exemplary embodiment of the disclosed subject matter.

FIG. 30 is a front end view of the implant in accordance with an exemplary embodiment of the disclosed subject matter.

FIG. 31 is a side view of the implant of FIG. 28.

FIG. 32 is a posterior view of the implant of FIG. 28.

FIG. 33 is a front, side isometric view from above of the implant of FIG. 28.

FIG. 34 is a rear, side isometric view from above of the implant of FIG. 28.

FIG. 35 is a front end view of the implant illustrated disposed on the skull of a subject in accordance with an exemplary embodiment of the disclosed subject matter.

FIG. 36 is a side view of the implant of FIG. 35.

FIG. 37 is a posterior view of the implant of FIG. 35.

FIG. 38 is a front, side isometric view from above of the implant of FIG. 35.

FIG. 39 is a rear, side isometric view from above of the implant of FIG. 35.

FIG. 40 is an illustration of the skull of a subject illustrated from below.

FIG. 41 is an isometric view from below of the implant of FIG. 30 disposed about the skull of the subject.

FIG. 42 is a representation of the implant disposed on the skull of the subject.

FIG. 43 is a representation of another embodiment of the implant illustrated on the skull of a subject.

FIG. 44 is a representation of another embodiment of the implant illustrated on the skull of a subject.

FIG. 45A is a front, side isometric view from above of an implant implanted on the skull of a subject in accordance with an exemplary embodiment of the disclosed subject matter.

FIG. 45B is a cross-sectional view of the implant of FIG. 45A.

FIG. 45C is front, side isometric view from above in cross-section of the implant of FIG. 45A.

FIG. 46A is a front, side isometric view from above of the implant of FIG. 45A.

FIG. 46B is a front, side isometric view from above of the implant of FIG. 45A implanted on the skull, with the skull shown as transparent.

FIG. 46C is a front, side isometric view from above of the implant of FIG. 45A implanted on the skull, with the skull shown as opaque.

FIG. 46D is a top view of the implant of FIG. 45A implanted on the skull.

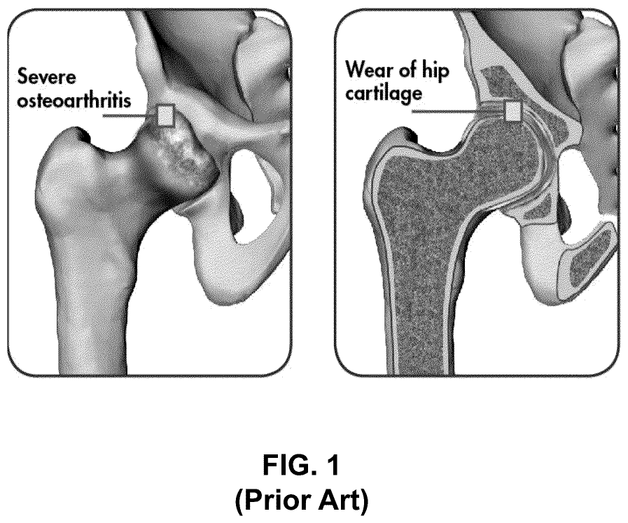

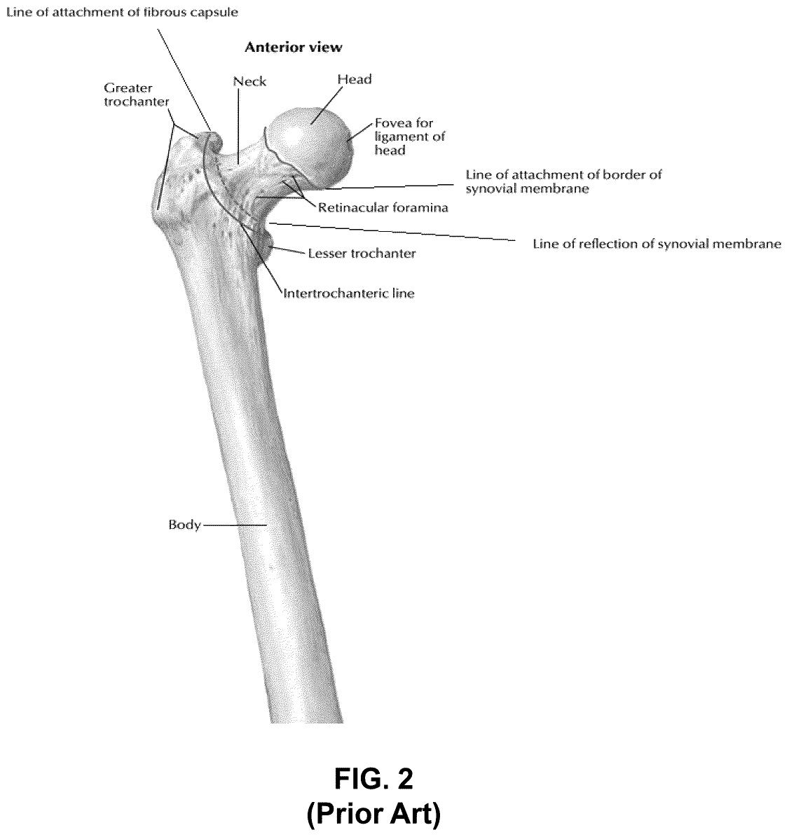

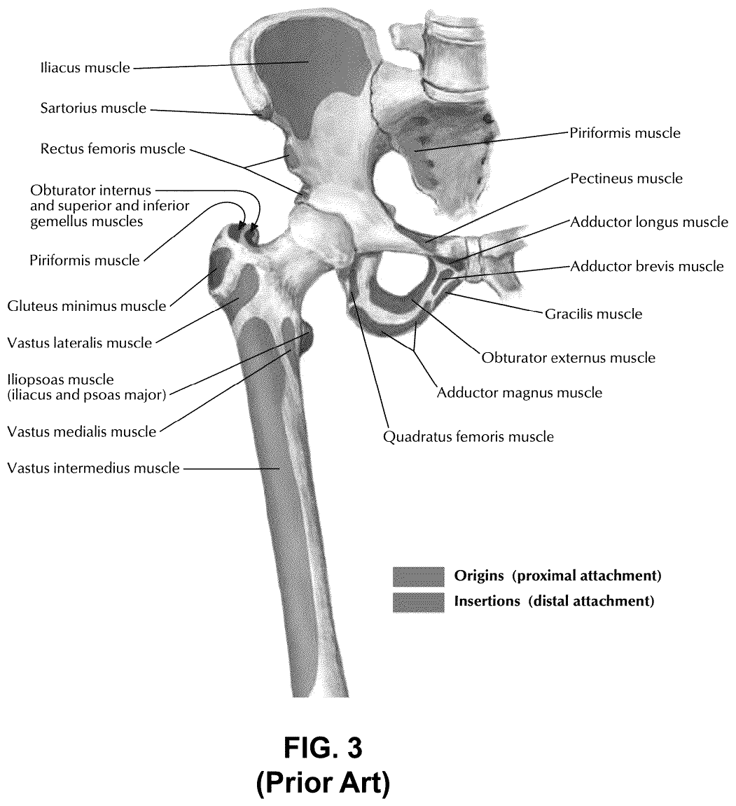

### DETAILED DESCRIPTION

Various detailed embodiments of the present disclosure, taken in conjunction with the accompanying figures, are disclosed herein; however, it is to be understood that the disclosed embodiments are merely illustrative. In addition, each of the examples given in connection with the various embodiments of the present disclosure is intended to be illustrative, and not restrictive.

Throughout the specification, the following terms take the meanings explicitly associated herein, unless the context clearly dictates otherwise. The phrases “in one embodiment” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments may be readily combined, without departing from the scope or spirit of the present disclosure.

In addition, the term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

As used herein, the terms “and” and “or” may be used interchangeably to refer to a set of items in both the conjunctive and disjunctive in order to encompass the full description of combinations and alternatives of the items. By way of example, a set of items may be listed with the disjunctive “or”, or with the conjunction “and.” In either case, the set is to be interpreted as meaning each of the items singularly as alternatives, as well as any combination of the listed items.

It is generally understood that hard tissue refers to bones and teeth. Pursuant to the present disclosure, the primary mode of attachment to the hard tissue is by non-infiltrating mechanical securement of the device to the tissue, as will be described in greater detail. Such non-infiltrating securement is made by a substantially non-invasive installation in the surgical procedure. This approach engages the exterior of the hard tissue and reduces contact with sensitive soft tissue structures underneath. Mechanical securement is generally provided by force closure (“firm grip” of the object) by appropriate choice of geometry of contacts (number, location, curvature and extent) defining the engagement surface of the device with the hard tissue and where motion of the object is resisted primarily by contact force (in practice, frictional forces also come into play). For example, when devices are secured to the skull, a non-infiltrating mechanical securement, via force closure, to exterior bone tissue is advantageous since the brain and dura are directly underneath the inner skull surface. In the case of hip replacement, a portion of the femur, e.g., the femoral head portion, is typically cut and removed. In the case of shoulder replacement, a portion of the upper end of the humerus may be cut and removed. For elbow replacement, a portion of the lower end of the humerus may be removed. To provide mechanical stability in these situations, some of which are high load bearing, it is preferred to secure an implant to the outside of bone via non-infiltrating mechanical securement rather than attachment to the spongy bone inside.

When referring to the non-infiltrating mechanical securement, it is understood that the primary method of securement is mechanical securement to the exterior of the hard tissue by conforming to each subject's unique anatomy. In addition, optional secondary attachment may be provided for additional stability of the device. However, such secondary attachment is intended to be minimally invasive and would typically not be deployed without the primary mechanical securement described herein. Optional secondary attachment may include the use of screws or adhesives. For example, screws used for secondary attachment have smaller diameter and shallower penetration into the hard tissue, when compared to screws having increased diameter and penetration depth when used to provide primary means of securement. Similarly, adhesives used for secondary attachment are provided in a quantity and thickness that would be substantially less than adhesives used for primary securement.

Force closure is possible for any solid body with a rigid end which applies to hard tissue, such as bone, having an end surface or a cut end (in the case of prosthetics). In some embodiments, securement relies on touch points or curvature-based force closure in at least one two-dimensional plane, but determination of points of securement could be done through other methods and algorithms. The devices described herein are mechanically attached to hard tissue, such as bone, having an end surface or a cut end (in the case of prosthetics) that will have directions of curvature such that over at least one curve, a total curvature greater than 180 degrees is traversed. For non-infiltrating mechanical securement, in some embodiments the device extends far enough to reach around the lower edges of the curve (beyond 180 degrees) to engage the underhang, thereby preventing two degrees of axial motion in that plane. In-plane rotation is generally prevented since biological hard tissue is irregular and does not have constant local curvature (e.g., circular symmetry), disallowing pure rotation in a plane. The same principle can be applied in other planes to obtain mechanical securement by wrapping around a sufficient amount of curvature if using curvature-based force closure but other methods using optimally placed rigid contact points also suffice, or in some directions, mobility of the device might be allowed for some applications.

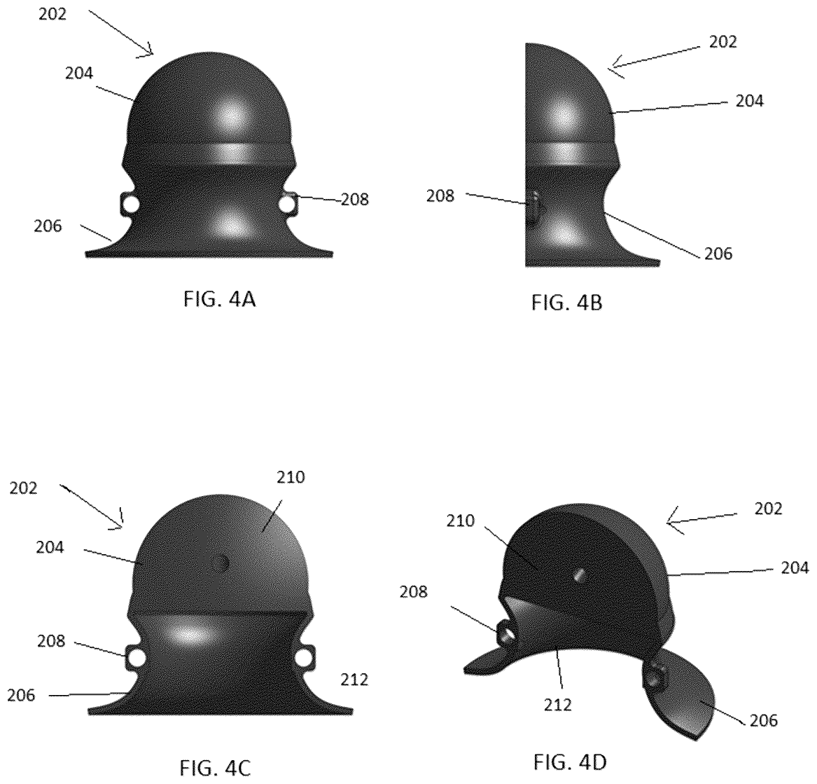

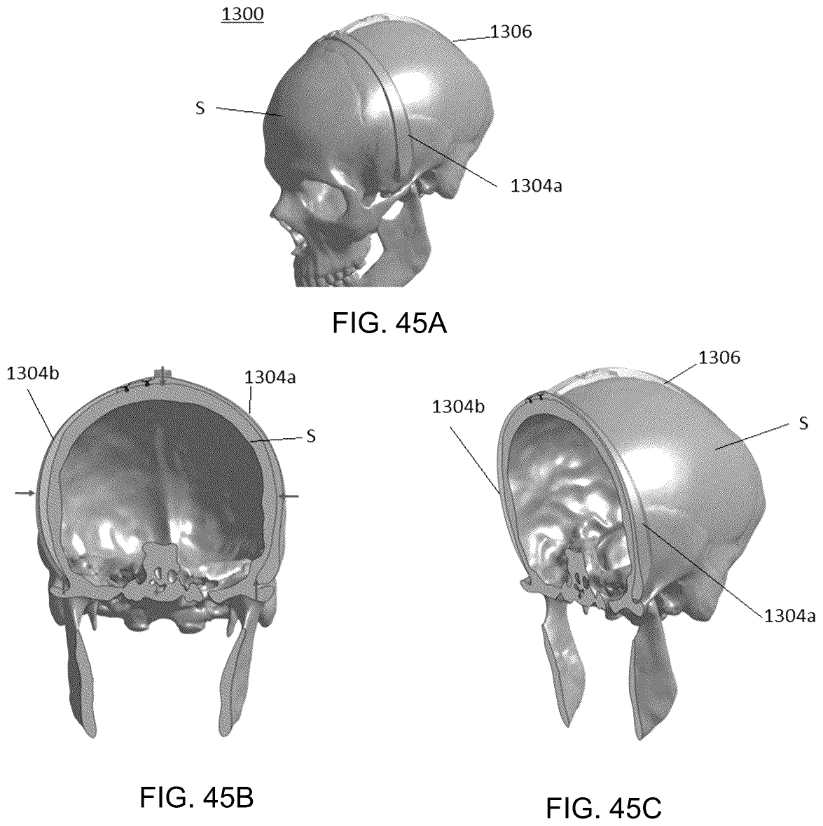

In embodiments of the subject matter, force closure mechanical securement in at least one two-dimensional plane is provided by a body portion having at least four contact points that provide an interface to an end portion of a curved surface that extends greater than 180 degrees in one or more planes. Three of the contact points are arranged such that they span greater than 180 degrees of total curvature of the end surface interface in the plane they define, and the fourth contact point is outside that plane. Contact points can be connected to form a continuous set of contact points, as seen, e.g., in the collar portion 206a/206 of the device 200 (FIGS. 4A-D and 5A-D) or the extended arms 1304a/1304b/1306 of the human neural implant 1300 (FIGS. 45A-C and 46A-D).

In some embodiments, where the available bone surface does not allow for one or more of these geometric constraints to obtain mechanical securement, one or more directions with unconstrained motion can be secured by placing cement or other bonding agent between the bone and the bone-conforming device's inner surface to secure the device while avoiding any additional infiltration of bone. Alternatively, screws, bolts, or infiltrating methods of securement could be used. In all cases, as a primary method of securement the device conforms to existing bone geometry for mechanical fixation using its aspects to constrain motion in some or all of axial and rotational direction and can be used in complementation with other methods of incidental, secondary securement such as screws and cement.

In some embodiments, the device is split into at least two portions in order to allow installation around the curved bone surface. Consequently, the component pieces themselves do not have greater than 180 degrees of curvature in at least one plane which then allows sliding pieces freely in the plane in at least one or more axes over the bone to surround it. Once the two or more pieces are engaged, they are fastened into a mechanically secure part that prevents motions by its conformance to some or all of the underlying bone or other hard tissue.

A conventional hip replacement technique is hip resurfacing arthroplasty (HRA). In HRA, force is directed into a bolt in the proximal part of the femur. Thus, for strength after mechanical insertion, there is a desire to use as long a bolt as possible which constrains how much of the head and neck of the femur can be removed, limiting space access during the surgical procedure and with downstream consequences for ball and socket size. Keeping a longer bolt in bone generally necessitates using a hollowed out ball over top which has the disadvantage of being brittle if made from preferred materials like ceramic. Instead, metals are used for the hollow ball which has the undesirable property of being a suboptimal bearing surface compared to other materials. This design constraint of a larger ball drives the need for larger artificial hip sockets to admit the larger diameter ball, and the socket also tends to be made of metal as the size-strength ratio of metal relative to plastic and ceramic allows for a thinner hip socket liner. The resulting metal-on-metal rubbing of ball and socket in conventional HRA has led to leaching of metals which causes inflammatory processes. By anchoring in and directing force into the softer, cancellous spongy bone in the proximal femur, this can be disadvantageous. Furthermore, the linear geometry of a bolt increases lever arm of the transferred force leading to fractures more often than in a long-stem total hip replacement arthroplasty (THA). Thus, THA remains the preferred method though it involves extensive modification of the body of the femur to insert a large shaft into its interiors.

HRA presents a higher degree of surgical difficulty than THA because the proximal portion of the femur neck is wide, and the surgeon is obliged to estimate where to insert the bolt. Furthermore, surgeons currently have to estimate length and angle to interface the ball with the socket to maintain leg length as well as achieve good force transfer.

A stereotyped ball and bolt geometry requires surgeons to modify femur head/neck to match the implant's surface increasing surgery time, and if done improperly, bone modification could lead to poor mechanics of the artificial hip joint.

Even in a successful implant, years of use lead to material wear which compromises rotation mechanics within the socket but also leeches metal particles that cause weakening of surrounding bone via inflammatory processes. Because parts are secured to bone, replacing them is not a trivial procedure and often the bone is not re-usable so a larger deeper implant shaft is needed if any revision is pursued or even a total hip arthroplasty (THA). Similar drawbacks can occur in the case of shoulder replacement, elbow arthroplasty, elbow radial head replacement, and other joint repair or replacement procedures.

In accordance with the disclosed embodiments, the device described herein can replace conventional implant components that infiltrate a bone in order to effect securement of the implant to the bone matter with components that be secured to a bone by non-invasively gripping the outer geometry of the cite of implantation. In the case of elbow arthroplasty, a TEA implant represents a conventional, invasive approach that would be eliminated using the presently disclosed system. The present system can also eliminate the requirement for the use of a humeral stem portion of an implant pursuant to shoulder arthroplasty. In further embodiments, the presently disclosed system replaces the component interfacing the artificial ball to the femur (and does not modify the natural or artificial socket; an artificial socket inserted into the acetabulum can be chosen accordingly in complementary material, size and shape using existing technologies). In any of the presently disclosed embodiments, the implant uses a precise bone surface model (reconstructed from standard medical imaging) of the neck-head geometry of the patient's bone, e.g., femur, humerus, radius, ulna, or the like, to obtain rigid, mechanical fixation by pure geometric fit-a tight collar that substantially conforms to at least a portion of the patient's bone end geometry in a fully constrained manner. The device includes a cap, either transitioning to or connected separately to a collar or sleeve. In certain embodiments, where appropriate based on the geometry of the portion of the bone that is adjacent to the bone end, the collar or sleeve can include curvature at its perimeter emulating the geometry of the head-to-neck transition of the bone. Proximal to the location of the joint, near where the neck flares to meet the head, this creates an inward curving geometry for the device to attach and limit axial motion (in both directions) along the axis of the neck. If desired, the device can be extended distally, in the outward direction toward the bone body where against the flare from the neck-to-body of the bone prevents proximal-to-distal axial sliding in a single axial direction (proximal-to-distal), distributing body force to the shaft of the bone. For example, in the case of a device according to the present disclosure being used in the context of a hip replacement, the device can be extended distally, in the outward direction toward the femur body where against the flare from the neck-to-body of the bone prevents proximal-to-distal axial sliding in a single axial direction (proximal-to-distal), distributing body force to the greater and lesser trochanter as well as the shaft of the femur. The irregular, varying local curvature of the femur neck creates interferences to any rotational force. Finally, preventing lateral (radial) movements is the fact that the presently disclosed device tightly wraps around the circular geometry of the quasi-cylindrical neck of the bone to which it is secured. In some embodiments, the device includes at least two interlocking pieces for installation and when clamped around the neck of the bone limits any unwanted motions, such as unwanted lateral motion in the case of a femur implant.

To give precise anatomical reference, in embodiments representing a femur implant, the device overlaps the line of attachment of border of synovial membrane (proximal flare of femur neck) and can extend to the line of reflection of synovial membrane (distal flare of femur neck)—but perhaps even as far as the intertrochanteric line distally.

In another embodiment, when the head or end of the bone is severely compromised from osteoarthritis or other damage and is to be removed below the flare to the neck, then the device could only engage the neck of the bone as well as any distal flare of the bone neck, where appropriate, such as in the case of the femur or humerus. This would potentially leave distal-to-proximal axial motion less constrained but need not and the device would still fit tightly and be relatively rigid. If any sliding is noted, then bone cement could be used at the proximal end of the bone to adhere the inside of the device. Alternatively, the implant could be made to extend further distal to go over a more distal portion of the bone anatomy.

For example, the implant can be made to extend over the trochanteric head of the femur. Here, the flares afforded by the greater and lesser trochanter would allow mechanical fixation in both directions axially when the two implant pieces are clamped together. However, this is a more complex approach as many muscle insertions occur on this part of the femur. The implant would include built-in windows that allow muscle tendons to pass through; during installation, the surgeon would have to cut the tendons, thread through the implant windows then suture back together. The advantage of wrapping the device around the trochanters is that this would give significant mechanical purchase across a large surface area of the femur.

An overall advantage of the device described herein is that it reduces compromise of the patient's bone by enclosing it rather than penetrating it. By preserving the bone as much as possible, the device allows more natural, physiological transfer of force through the proximal bone which may improve activity level after implantation, benefiting the patient. For example, in the case of the femur, the device can allow more natural, physiological transfer of force including through the metaphysis and the calcar growth plate. Conversely, benefiting the implant, this more natural transfer of force and distribution across a broader implant collar may prolong the life of the implant materials and limit leak of metal particles. Improved mechanical robustness could admit a wider catalog of materials including usage of more forgiving, biocompatible materials like titanium as cobalt-chrome is currently used for its hardness but also tends to release inflammatory metal particles. Softer materials may wear down, but can be replaced given the reversibility of the clamp installation and modular variants of the design, with the benefit that softer materials may be more biocompatible and cause less inflammation. Different materials can also be combined in this design. A metal or strong, load-bearing material can be chosen for the collar, and this component would require 3D printing or CNC machining to match the patient's bone geometry. In tandem, where used, a simple ball geometry (such as in the case of a femur or upper humerus implant) can be generally made from a smooth material like ceramic or plastic to provide a suitably smooth bearing surface to slide within the corresponding socket. As opposed to the bone-conformed collar primarily around the bone neck, a spherical ball (replacing the femoral or humeral head) could be simply manufactured and not require precise machining which lowers production cost and expands the range of material options. Modularity allows replacing the sliding ball portion which may experience wear and tear while keeping the collar portion in place allowing it to osseointegrate with underlying bone. Attaching or fastening different pieces to each other can be done by a variety of methods including bolts, glues, acrylics, cements, or simple press fit; all interfaces between pieces can be made on internal aspects of the device to maintain a smooth external profile, or through attachment on the external surface, and not be to the bone as the bone securement is simply done by closing the pieces together around the bone. For added strength of the device in the collar portion overlying the bone neck, any or all of these additional measures can be taken: the sleeve of the collar portion could be made thicker, external fastening points between the pieces could be added near the base of the collar, or screws or cement can be used to fasten directly into bone of the neck of the bone.

Comparing to hip resurfacing arthroplasty (HRA), total elbow arthroplasty (TEA), or total shoulder arthroplasty, which attempt to minimize bone modification, embodiments of the implant present a number of added advantages. First, the installation is much simpler as it requires more minimal bone remodeling by the surgeon to fit the device (e.g., the implant is made to match the patient's anatomy). A single planar cut of the bone, such as the femur or humerus head, is done at a height and angle that can be determined by a 3D-printed guide, and the present design does not preclude other geometries of modifying the bone as the device geometry is made to match the target geometry the surgeon desires. The height of the cut can be tailored to some degree by the surgeon based on considerations such as the amount of compromised cartilage that needs removal, the amount of surgical access that may be needed (cut more distal), and the amount of bone neck to leave for mechanical purchase stability (cut more proximal). The angle of the cut could be adjusted to make sliding the pieces together easier in the surgery. Then the device with cap and sleeve is simply slid over the remaining head and neck of the bone. The device is self-centering as it precisely conforms to the patient's bone anatomy. Given the asymmetries of bone, there will be exactly one location for a conforming fit that allows no relative movement. Thus, the surgeon does not need to estimate how to anchor the device (location, angle) or reshape underlying bone; this removes multiple decision points and provides only one correct solution.

Besides making the surgery shorter and more error-proof, the device has long-term benefits. Because it envelops the neck and remaining head of the bone, it provides a complete, distributed mechanical interface which is ideal for this application. This is also true in the case of a hip implant, which represents a load-bearing application. As compared with hip resurfacing arthroplasty (HRA), total elbow arthroplasty (TEA), or total shoulder arthroplasty, the present approach can be applied without use of a bolt that goes into the center of the neck or shaft invading the center of the body of the bone. In these prior approaches, the localized axial interface of bolt distributes force primarily along one dimension which can result in fracture if not properly installed or over the course of wear and tear or if bone remodeling does not allow for ingrowth. By invading the bone, a bolt or shaft also creates opportunity for infection and inflammation eventually resulting in implant failure. The externally mechanically secured implant does not create any openings in the bone besides replacing the damaged bone head, a necessary bone modification. By distributing force across multiple directions on the hard outer surface (compact bone) of the bone, fracture is less likely than a poorly leveraged bolt interface into spongy, cancellous bone. The implant does not depend on bone remodeling for maximal strength, for example, as in a HRA or THA insertion, TEA implant insertion, or shoulder arthroplasty insertion. It works immediately, having near maximal efficacy by relying mainly on the existing bone geometry, though bone remodeling could cause further integration into the device.

Exemplary embodiments of the devices are described herein. FIGS. 4A-4D illustrate an exemplary embodiment of device 200. In some embodiments, the device 200 is symmetric. Accordingly, device 200 includes two substantially identical components 202 secured together about the bone. Device 200 is two pieces 202 that clamp along the midline and are secured, e.g., by using a diagonal bolt not shown. FIGS. 4A-D illustrate one of two substantially symmetrical components 202. Each component includes a substantially spherical head or ball portion 204 transitioning to a sleeve or collar portion 206. When in the form of a ball, head portion 204 is typically solid and has a generally half-spherical exterior contour. Head portion 204 also has an interior surface 210 that mates with the interior surface 210 of the mating component 202 as will be described below. The embodiment depicted in FIGS. 4A-D is particularly suited for hip replacement (i.e., as the head of the femur) or shoulder arthroplasty (for the end of the humerus). In other embodiments, depending on the end use, ball portion 202 is replaced with a flat portion, concave portion, or other portion that is shaped such as to (i) approximate the end of the particular type of bone that it is intended to replace, or (ii) represent a reverse member of the joint (such as to represent the socket member of a ball-and-socket joint). For example, in the case of elbow arthroplasty, the device may include a substantially flat portion in place of the ball portion (for radial head replacement) or a less drastically rounded portion in place of the ball portion (for humeral head replacement). In another example, the device may include a concave, cup-shaped portion so that the implant can function as the socket in a ball-and-socket type joint, such as in the case of reverse shoulder arthroplasty or reverse femoral arthroplasty. Collar portion 206 has a concave interior surface 212 that conforms to the exterior surface of the bone adjacent to the end of the bone, such as the neck portion of the femur or humerus. In some embodiments, the interior surface 212 is fabricated to conform to the entire exterior surface of the bone, e.g., by 3D printing. In some embodiments, the interior surface 212 is fabricated to conform to a portion of the exterior surface of the bone. In some embodiments, the exterior surface of the bone is the natural surface. In some embodiments, the exterior surface has been scraped, smoothed or otherwise reshaped to remove irregular projections or to provide a geometric shape, such as a rectangular cross-section. The two components 202 of the device 200 are secured together. In some embodiments, the components 202 are secured by bolts passing through the securement points 208. Typical materials for this device are titanium, cobalt-chrome, ceramic, and polyethylene and other plastics and metals could be considered. In some embodiments, the collar is titanium. Custom shapes can be additively manufactured, and titanium is known for high compatibility with bone. In some embodiments, the head (e.g., the ball or the alternative to the ball) is ceramic. For example, ceramic provides a smooth, bearing surface for articulating with artificial hip or shoulder socket liners, typically made of polyethylene. In embodiments where the ball and collar are not separate, then the preferred single material for manufacturing them is a metal, titanium or cobalt-chrome.

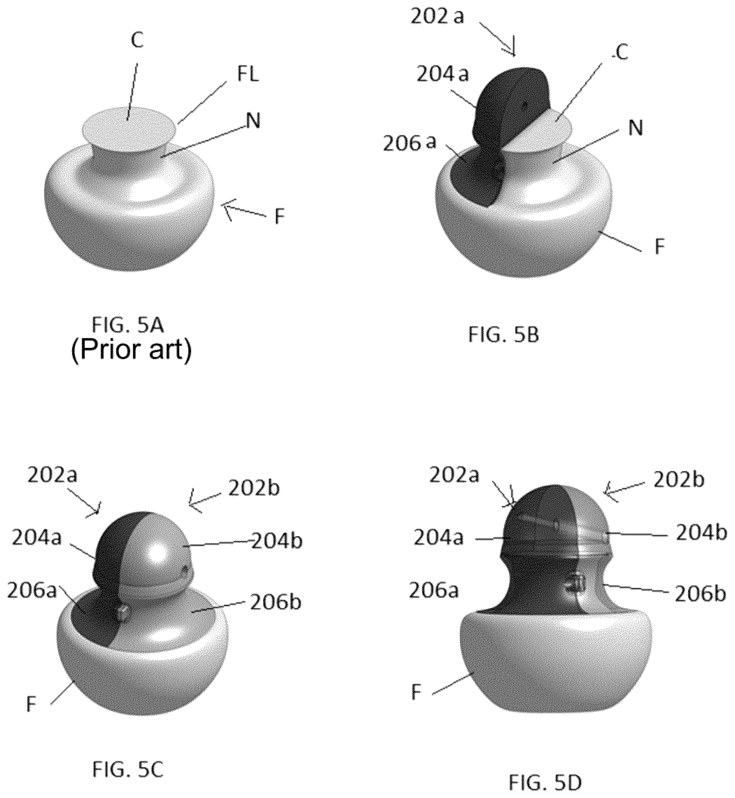

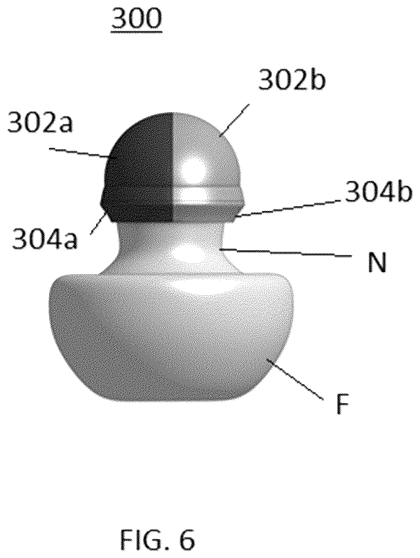

FIGS. 5A-5D illustrates an exemplary stepwise installation of the device 200 of FIGS. 4A-4D over bone. FIG. 5A illustrates a simplified view of bone F (such as a femur or humerus) including the neck portion N and surface C representing the exposed surface after removal of the head portion of the bone F. Proximal to the head, a portion FL of the neck N flares to meet the head (which has been removed). FIG. 5B illustrates installation of a first component 202a of the device 200. The component 202a is placed such that the ball portion 204a is placed on surface C and the interior surface (not shown) of the collar 206a surrounds a portion of the neck N of the bone F. FIGS. 5C-5D illustrate that second component 202b is placed over the bone F in a similar fashion as component 202a, e.g., ball portion 204b is placed on surface C and the interior surface (not shown) of the collar 206b surrounds a portion of the neck N of the bone F, such as a femur or humerus. The implant is fastened with screws not shown in the figures. A similar procedure may be used for installation of the device 200 (for example, with a head 204 having a configuration other than a ball) onto a different bone, such as onto the humerus (top end or bottom end), radius, ulna, phalanges, metacarpal, or another bone that is well-suited for accepting an implant according to the present disclosure.

As discussed above, the device 200 uses a precise bone surface model of the neck-head geometry of the patient's bone F (such as a femur or humerus) to obtain rigid, mechanical fixation by pure geometric fit-a tight collar 206a/206b substantially conforms to at least a portion of the geometry of the patient's bone neck N in a fully constrained manner. Near where the neck flares to meet the head FL, the neck N has an inward curving geometry for the interior surface of the sleeve 206a/206b to attach and limit axial motion (in both directions) along the axis of the neck N. The irregular, varying local curvature of the bone neck creates interferences to any rotational force. Finally, preventing lateral (radial) movements is the fact that our device tightly wraps around the circular geometry of the quasi-cylindrical bone neck.

The head size and collar indentation are designed in accordance with exemplary embodiments. For example, if a small head size (e.g., small ball size) is desired, then the implant collar can be made to have a narrowing before expanding out. This narrowing/neck portion would have a length to accommodate the smaller head radius and preserve patient bone length. Other geometric adjustments can be used depending on the bone on which the prosthesis is being secured, and depending on the configuration of the head.

Further embodiments include modifications to the collar. In some embodiments, a short collar for minimizing interference with surrounding tissue is used. FIG. 6 illustrates a device 300 having symmetric components (similar to FIGS. 4A-4D) having head 302a/302b with a shorter collar 304a/304b. In a version of this embodiment that is adapted for use on a bone other than the femur or humerus, for example, the head in the device of FIG. 6 may have a different configuration, e.g., flat, concave, etc., as described supra.

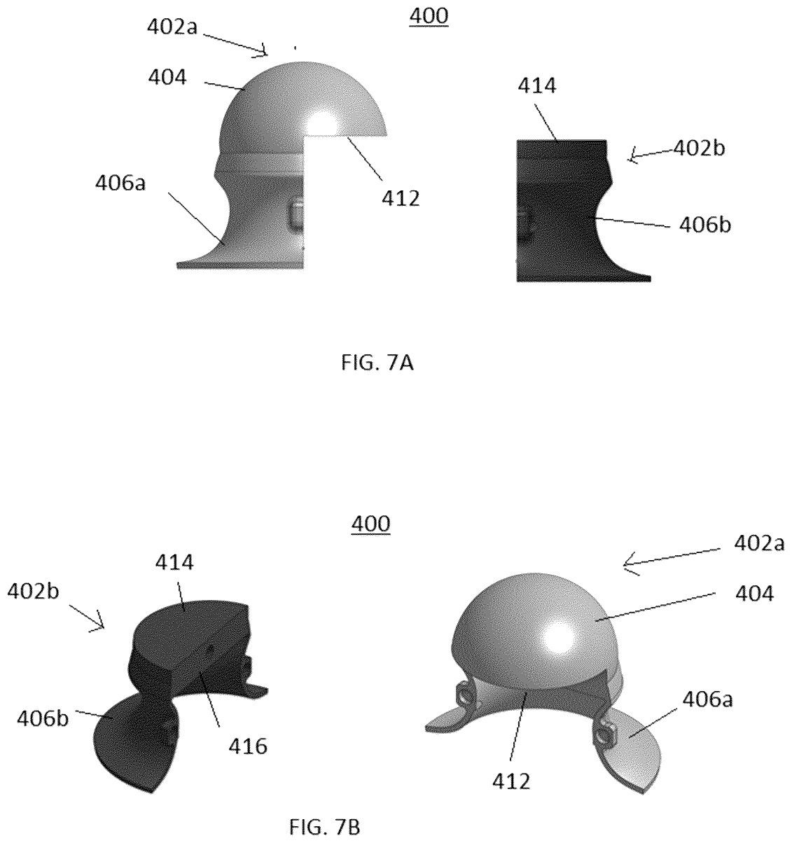

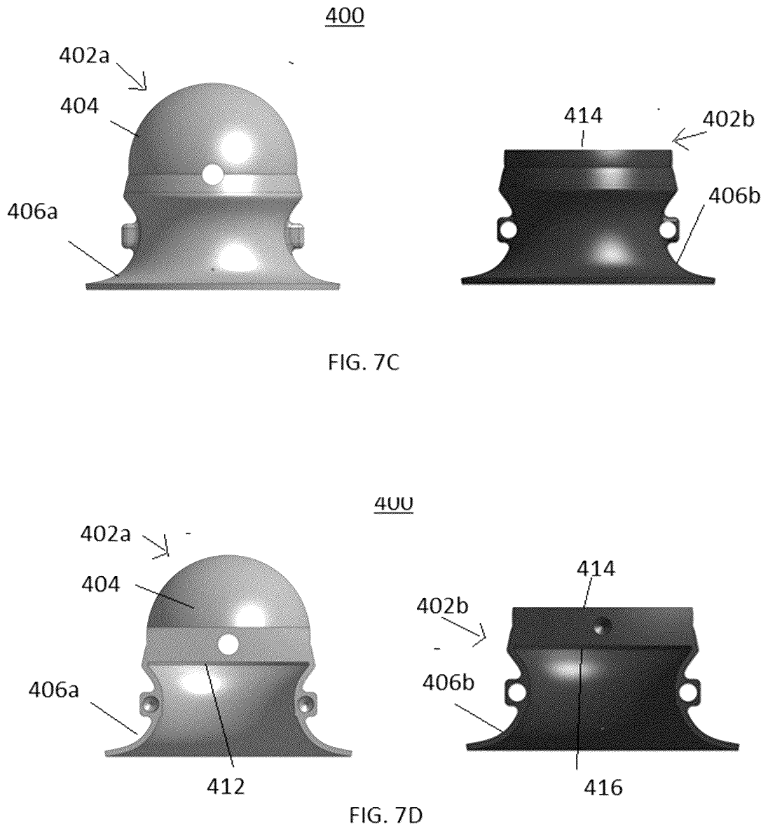

As illustrated in FIGS. 7A-7D, the device 400 is asymmetric in some embodiments, in which the head 404 comprising a flange- and ball-type cap is integral with the first collar portion 406a to form the first component 402a, and the second collar portion 406b forms the second component. A majority of the overlying head/cap 404 is part of one component 402a to minimize seam length in the joint, such as in a hip socket. The second component 402b, which includes a flange-like overhanging cap bounded by upper surface 414 and lower surface 416, interlocks using a horizontal fastening mechanism. As illustrated in FIGS. 7A-7D, component 402a includes ball 404 and collar 406a. Component 402b includes collar 406b. When component 402a is secured to component 402b, the lower surface 412 of ball 404 mates with the upper surface 414 of component 402b. The lower surface 416 of component 402b caps surface C of bonc F, i.e., is positioned on the surface C of the bone F from which the end (e.g., head portion in the case of the femur or humerus) has been removed. This configuration may be adapted for use with a different head configuration, e.g., flat, concave, etc., as described supra.

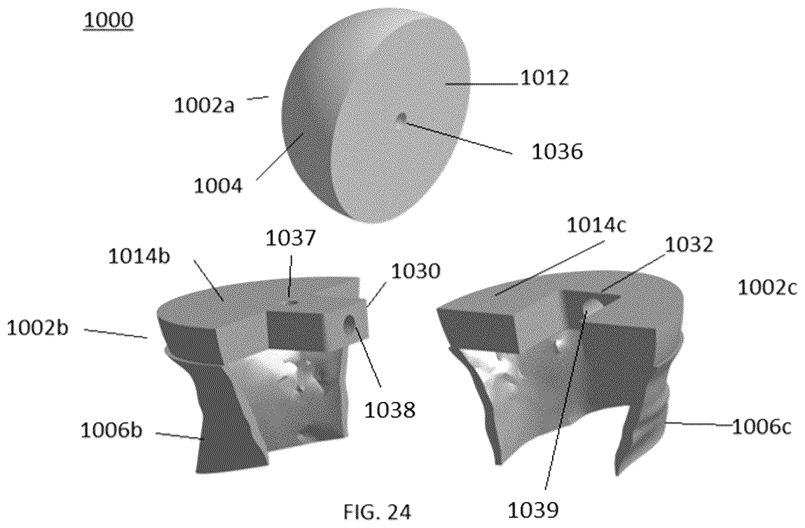

As the term is used herein, a “cap” portion of the prosthesis is any component that overlays a bone end when the prosthesis is secured thereto. The term is sometimes used interchangeably with “head”, although the “head” preferably refers to the distal-most element of the prosthesis that participates in the joint. In the embodiment shown in FIGS. 7A and 7B, the cap includes two distinct elements: the flange bounded by upper surface 414 and lower surface 416 of component 402b, and the flange/ball element 404 bounded by lower surface 412 and the upper hemispherical surface shown in FIG. 7B. In some embodiments, such as that which is depicted in FIG. 24 (described more fully infra) the cap can include three elements (in the figure, the three elements are the flange bounded by surface 1014b, the flange bounded by surface 1014c, and hemisphere 1004) that are assembled together during securement of the prosthesis to the bone end.

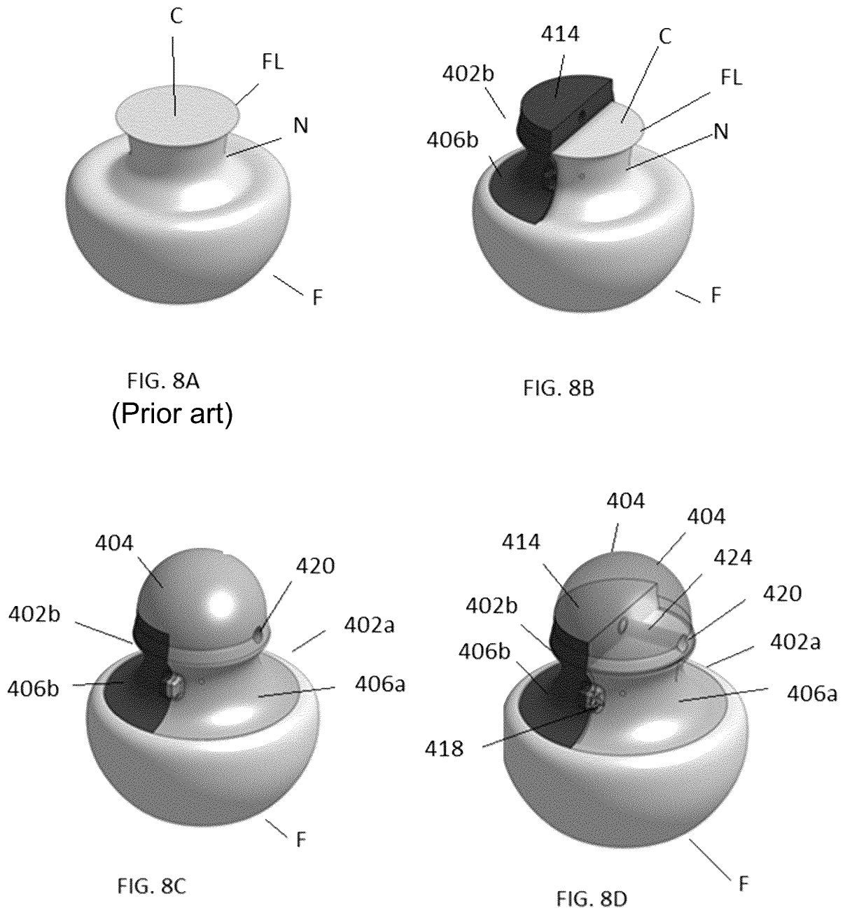

FIGS. 8A-8D illustrates an exemplary stepwise installation of the device 400 of FIGS. 7A-7D over bone F. FIG. 8A illustrates a simplified view of bone F, for example, a femur or upper end of a humerus, including the neck portion N and flared portion FL and surface C representing the exposed surface after removal of the head portion of the bone F. FIG. 8B illustrates installation of a first component 402b of the device 400. The component 402b is placed such that the lower surface 416 (not shown) is placed on surface C and the interior surface (not shown) of the collar 406b surrounds a portion of the neck N of the bone F. FIGS. 8C-8D illustrate that second component 402a is placed over the bone F, e.g., the lower surface 412 of head portion 404 (which in this embodiment represents a hemisphere or “ball”) is placed partially on surface C and partially on upper surface 414. The interior surface (not shown) of the collar 406b surrounds a portion of the neck N of the bone F. The implant is fastened with screws. For example, screws may be used to connect components 402a and 402b and connecting points 418. An aperture 420 may be provided in head portion 404 and upper portion of 402a to receive a bolt 424 to further secure the components.

The head size and collar indentation are designed in accordance with exemplary embodiments. For example, if a small head (e.g., ball) size is desired, then the implant collar can be made to have a narrowing before expanding out. This narrowing/neck portion would have a length to accommodate the smaller head radius and preserve patient bone length. The illustrated devices incorporate a ball that matches the size of the original femur head and would work for large artificial sockets used in hip replacements.

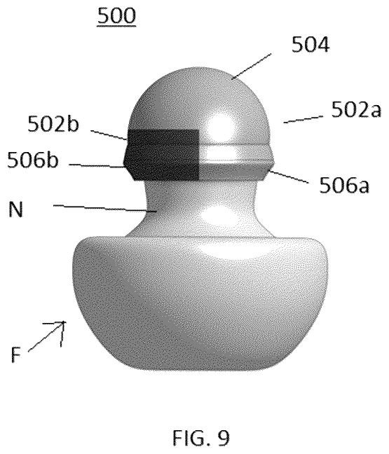

FIG. 9 illustrates a device 500 having asymmetric components 502a and 502b (similar to FIGS. 7A-7D) having a head portion 504 (in this embodiment, a hemisphere) on one of the two components (502a) with a short collar design 506a/506b. The short collar 506a/506b that only wraps the proximal head-neck N will give mechanical fixation with a small footprint so that ligaments and tendons, for example, those of the hip inserting at the femur, are not interfered with and making the installation surgery more straightforward. If a significant part of the head-neck is preserved in a patient, then a short collar is certainly feasible and potentially more indicated.

In some embodiments, a long collar provides added force distribution. Extending the collar to have purchase with the distal head-neck flare will create more distribution of axial forces. However, the added length may begin to encroach on soft tissue structures such as ligament insertions or inserting tendons.

In some embodiments, a long collar is provided for a compromised neck-head flare. If the head of the bone needs to be fully removed in a patient, this limits mechanical purchase of the implant at the proximal bone neck, requiring extension in the distal direction. However, a version with a long collar engaging the distal neck-body flare would limit axial motion in one direction. The proximal ball end could be screwed or cemented to bone to limit axial sliding or if the sleeve is a very tight fit to the remaining neck's geometry this may also adequately limit axial slide.

The implant is fastened in accordance with exemplary embodiments. Bolt heads can be cemented over or custom rivets can be made to create a smooth continuous surface with the implant. Any fastening points along the neck of the bone can be moved distally to avoid interference with the anatomical joint of which the bone is a member, such as the hip socket. Distal placement of fastening would benefit from a longer collar. In some embodiments, instead of bolts, implant pieces can be press fit and/or cemented to each other which avoids introducing screws. Cement is not used directly on bone, it is used to fasten implant pieces together. Any gaps from bone to implant could be filled with standard cement agents. If strength is a concern even with a good implant fit, anchoring screws or cement to the outer bone could be used but these would serve as a secondary mechanism for the implant's security. Additionally, the implant sleeve along the bone neck could be made thicker to create greater structural integrity, and being distal to the anatomical joint, this would minimally interfere with the anatomical joint.

The location of the implant joint (i.e., location at which the respective components are coupled) between the pieces of the implant can vary depending on surgical and mechanical considerations. In an exemplary embodiment, the split goes up to the neck and stops short of the head so that the head is one continuous piece or the head (e.g., hemisphere or “ball”) is a separate piece altogether, and the seam is short in the axial direction but extends laterally. In another embodiment, the entire implant is split in half from cylindrical neck to head, but then fastening would be needed across the axial extent creating a longer vertical seam with no lateral seam. The implant head diameter and geometry nearing the flare of the femur neck would be chosen according to space constraints such that the head can function properly without interference within the anatomical joint. This may require choosing a smaller head and allowing for some offset from the collar portion of the implant and/or removing enough of the neck to reach the narrow diameter portion which would then allow a smaller diameter head and sleeve. Alternatively, in the case of a hip prosthesis, if the implant wall is thin enough or the hip socket is made larger and clearance is possible near the socket, then a larger ball more similar to the original femur head is possible. In some cases, additional remodeling of the bone neck/head may be necessary by the surgeon to compensate for any issues with fitting the implant. Slightly different implant sizes can also be manufactured to allow for tolerance in the manufacturing process giving precise sizing options during the surgical procedure for installation.

The implant can be split along various planes into two or more pieces. One consideration is along which direction to cut to allow for the pieces to individually slide around the cross-sectional contour of the bone. Also, the direction of sliding the pieces onto the cut bone can be chosen for surgical convenience. For sliding pieces across front to back, this would involve a cut along the anterior-posterior midline of the bone head. For sliding pieces from medial to lateral, this would involve a cut along the medial-lateral axis of the bone head. Alternatively, the device could be cut into more pieces if that presents advantages for installation.

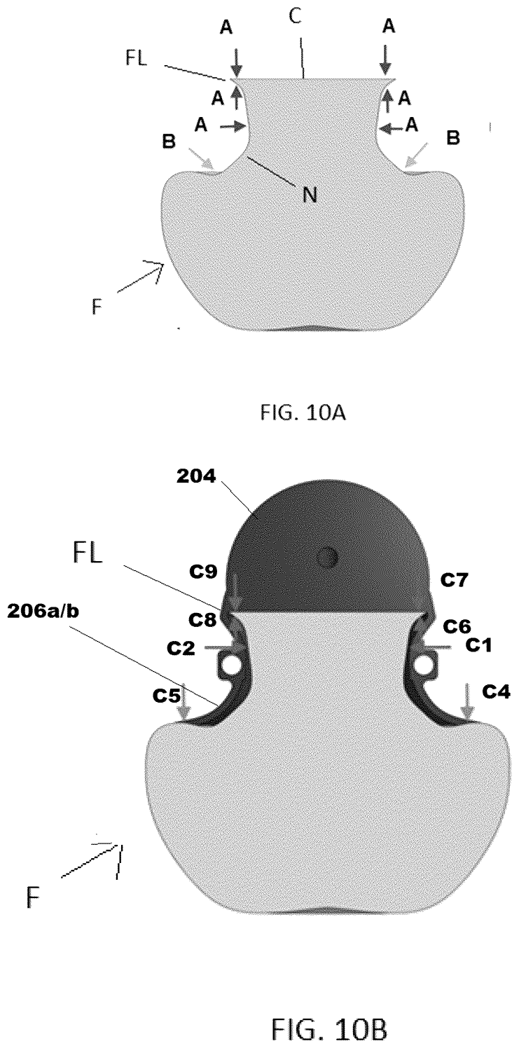

FIG. 10A illustrates a genericized version of the end of a bone F and the contact points of the implant on the bone. The flare FL in ends of neck N of bone F creates points for securing rigidly to the neck N of the bone F (arrows “A”). As shown in FIG. 10B, the device 200 wraps around the overhang created in the flare portion FL, i.e., transition from neck N to head of bone F. Force closure is provided by the collar 206a/b, having at least three contact points arranged such that they span greater than 180 degrees of total curvature of the end surface interface in the plane they define. (Two contact points “C1” and “C2” are shown in FIG. 10B). Extending the implant toward the body creates additional contact points out of the plane of the three contact points, above, to prevent axial motion (contact points “C4” and/or “C5”). In case of imperfect fit to bone contour, redundant contacts (arrows “C6,” “C7,” “C8,” and “C9”) provide stability, or bone cement can be used to fill gaps. Wrapping around the neck-body transition can be done if the neck-head transition (arrows “A”) is not possible. Moreover, in-plane rotation of the collar about the neck N is generally prevented since biological hard tissue is irregular and does not have constant local curvature (circle), disallowing pure rotation in a plane. It is understood that the principles described herein regarding externally surrounding a femur, for example, have applicability to any hard tissue.

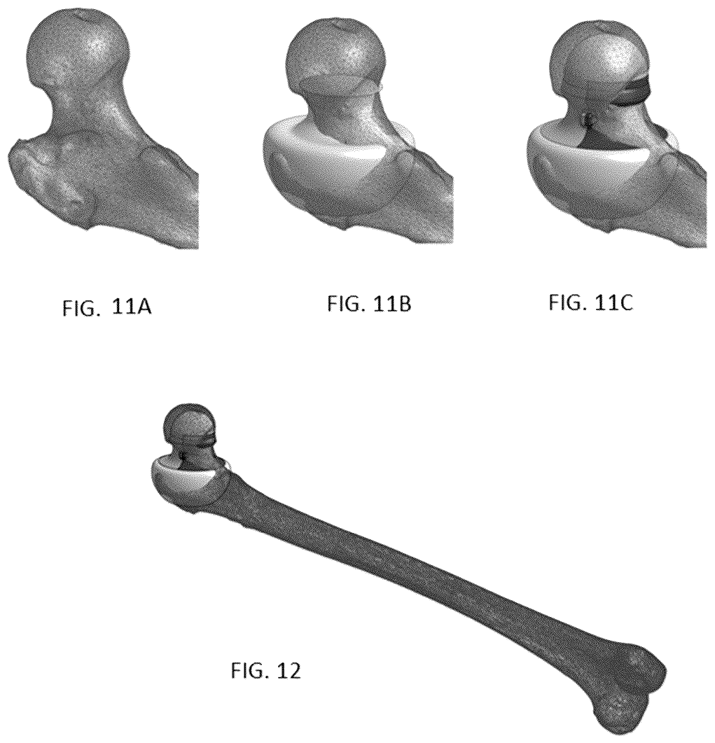

FIGS. 11A-C and 12 illustrate a technique for manufacturing and installing the implant, in this case, onto a femur. FIG. 11A illustrates the head and neck of the femur in need of replacement. FIG. 11B illustrates an overlay of a simple geometric model after the surgical cut. FIGS. 11C and 12 illustrate an overlay of the implant on the head-neck model.

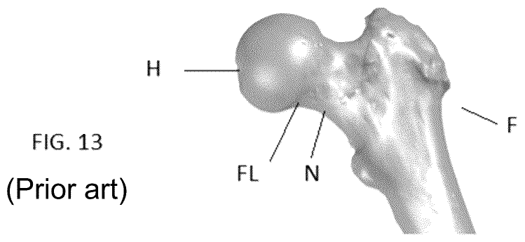

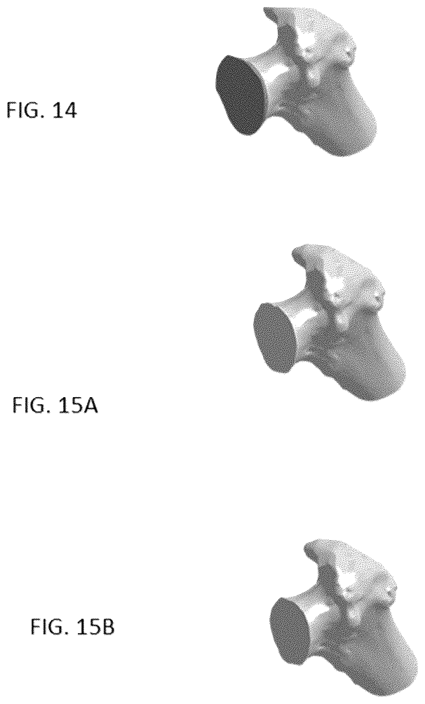

FIG. 13 illustrates the physiology of hip joint of the femur F. FIGS. 14 and 15A-15B illustrate the ball portion of the hip joint removed at several possible location. The physician may make a determination, based on the condition of the patient's bone structure to remove the ball joint at various points along the neck portion, and allow the flared portion to remain or be removed as indicated. The same principles can apply to any other bone onto which there is a need for installing an implant according to the present disclosure.

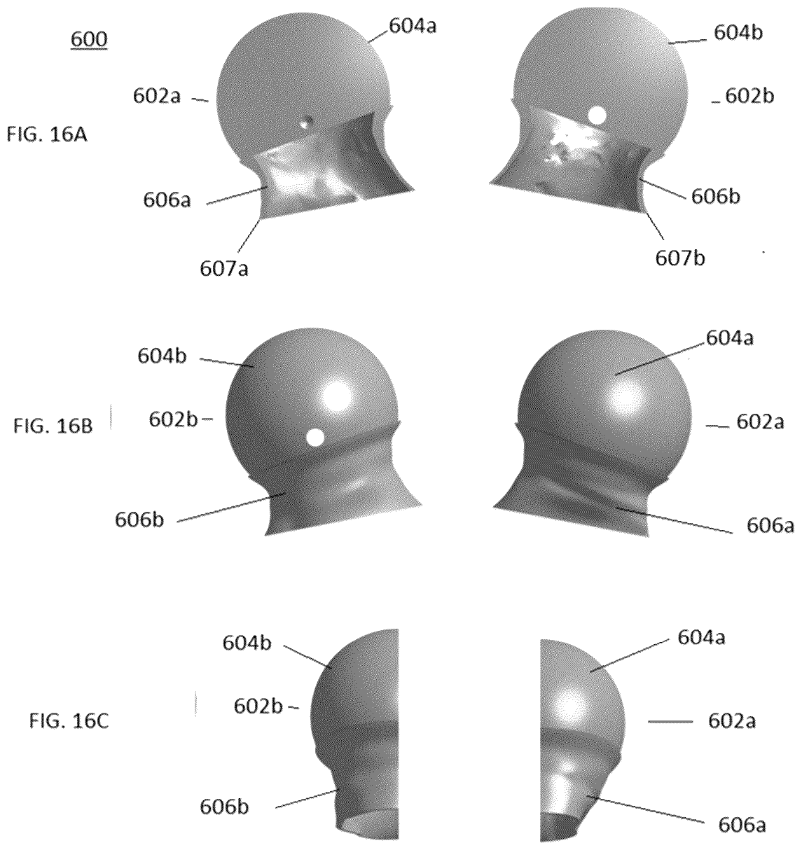

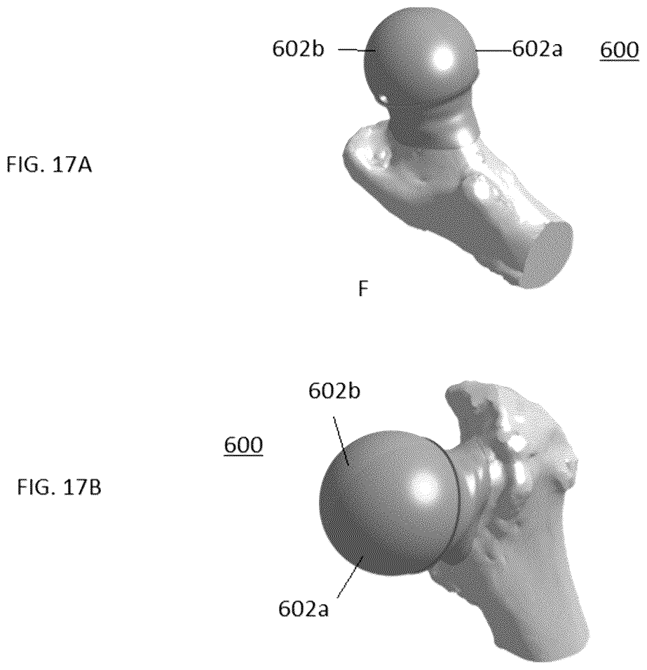

FIGS. 16A-16C illustrate an exemplary embodiment of device 600 that includes a head 604a and 604b that is adapted for participation in a ball-and-socket joint, such as at the hip or shoulder. Device 600 is substantially the same as 200, illustrated above in FIGS. 4A-4B, with the differences noted here. This device is conformant to the allowable neck of the bone within the bone capsule that spares any connection points for tendons and ligaments more distally. The cut along the coronal plane anatomically produces anterior and posterior halves which is favorable to an anterior surgical approach for sliding the pieces. Device 600, like device 200 includes a pair of symmetric components 602a and 602b. Each component 602a and 602b includes a ball portion 604a and 604b and a collar portion 606a and 606b. When components 602a and 602b are joined, collar 606a/b provides mechanical securement to the neck N of the bone F, as shown with respect to a femur in FIGS. 17A and 17B. This neck portion 606a/b is configured to be longer and includes an outward flared portion 607a/b (as shown in FIG. 16A) in order to engage a substantial length of the neck N to provide stable securement to the bone. The same principles can apply to any other bone onto which there is a need for installing an implant according to the present disclosure.

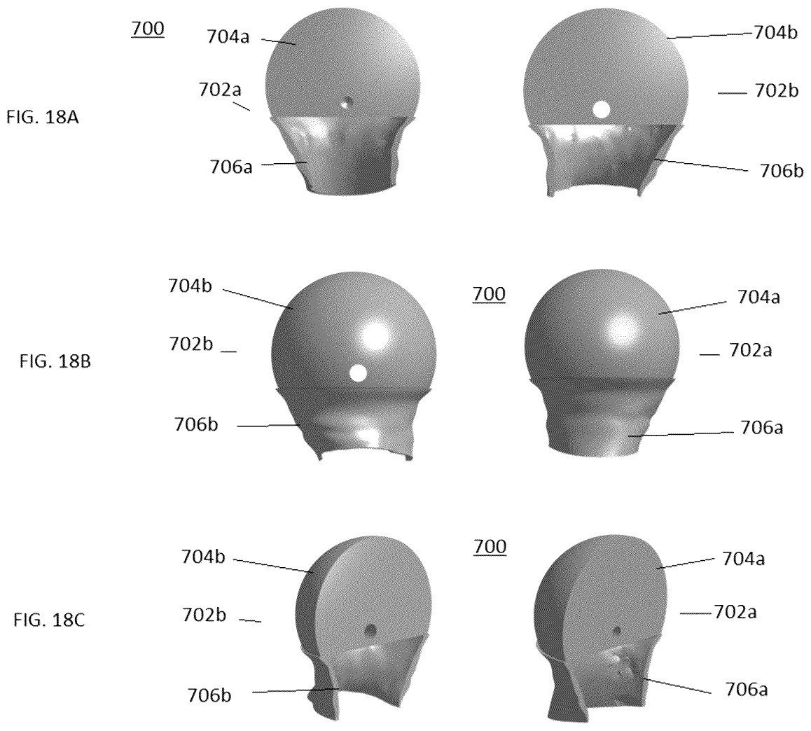

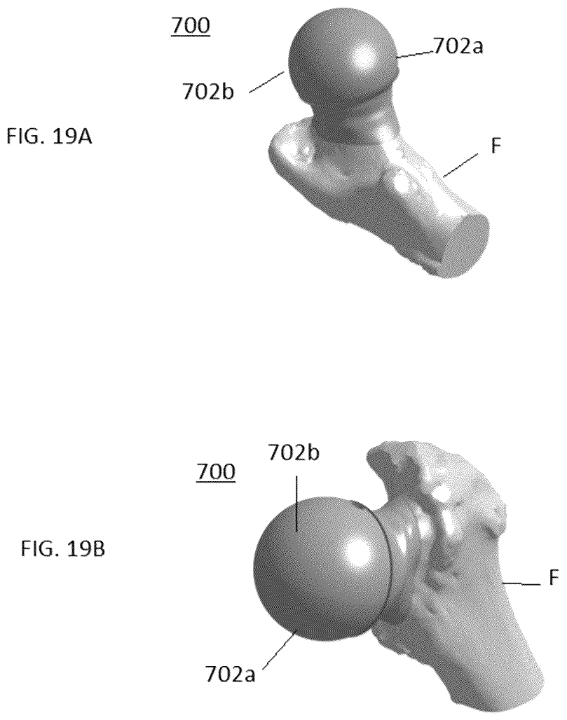

FIGS. 18A-18C illustrate an exemplary embodiment of device 700 that is adapted for implantation onto a bone that participates in a ball-and-socket joint, such as at the hip or shoulder. Device 700 is substantially the same as device 200, illustrated above in FIGS. 4A-4B, with the differences noted here. This device is conformant to the allowable neck of the bone, e.g., within the hip capsule, that spares any connection points for tendons and ligaments more distally. The cut along the horizontal plane anatomically produces superior and inferior halves which allows for securement of the pieces from the superior side during surgery and cuts the pieces along a plane their sliding over bone is simpler and the highest curvature, most constraining (strongest) part of the collar is not interrupted by the seam introduced by the cut. Device 700, like device 200, includes a pair of symmetric components 702a and 702b. Each component 702a and 702b includes a head portion 704a and 704b in the form of a hemisphere/ball and a collar portion 706a and 706b. When components 702a and 702b are joined, collar 706a/b provides mechanical securement to the neck N of the bone F, as shown in FIGS. 19A and 19B with respect to a femur. This neck portion 706a/b is configured to be longer in order to engage a substantial length of the neck N to provide stable securement to the bone. The same principles can apply to any other bone onto which there is a need for installing an implant according to the present disclosure.

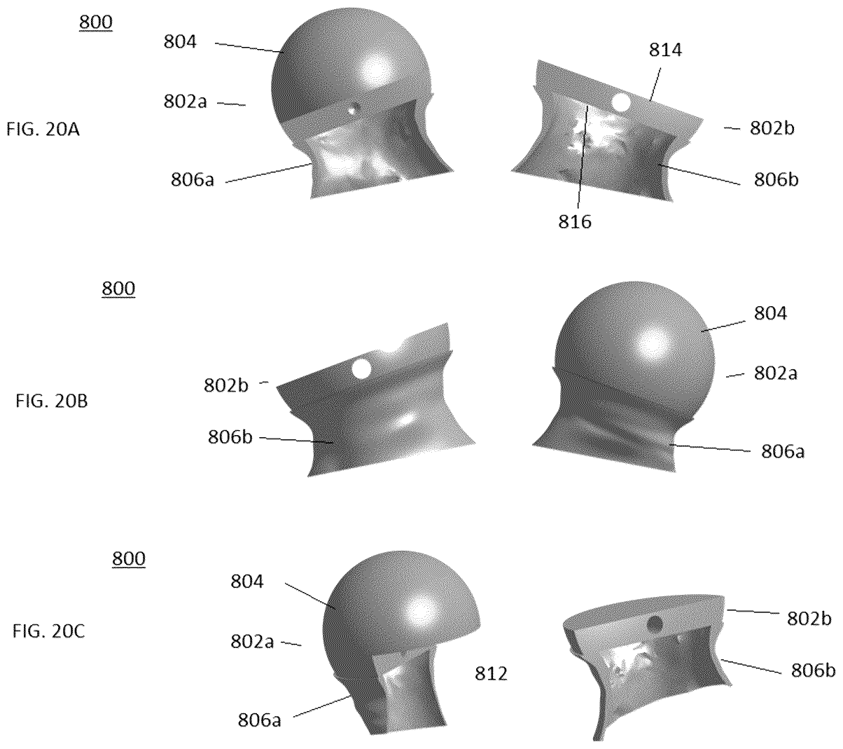

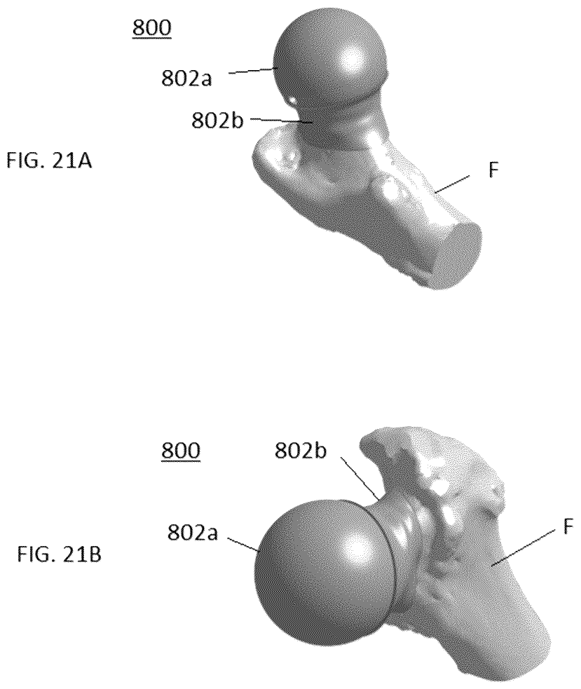

FIGS. 20A-20C illustrate an exemplary embodiment of device 800 that is adapted for implantation onto a bone that participates in a ball-and-socket joint, such as at the hip or shoulder. Device 800 is substantially the same as device 400, illustrated above in FIGS. 7A-7D, with the differences noted here. Like device 600, illustrated in FIGS. 16A-16C, this device 800 is cut along the coronal plane anatomically producing anterior and posterior halves which is favorable to an anterior surgical approach for sliding the pieces. The cut is interrupted immediately above the proximal end of the neck to avoid a cut along the ball except near its base. The reduced seam on the ball presents a smoother surface for articulation in the socket. Device 800, like device 400, is asymmetric in some embodiments, in which a majority of the overlying ball 804 is part of one component 802a to minimize seam length in the bone socket. The second component 802b interlocks using a horizontal fastening mechanism. As illustrated in FIGS. 20A-20C, component 802a includes ball 804 and collar 806a. Component 802b includes collar 806b. When component 802a is secured to component 802b, the lower surface 812 of ball 804 mates with the upper surface 814 of component 802b. This neck portion 806a/b is configured to be longer and includes an outward flared portion 807a/b (as shown in FIG. 20A) in order to engage a substantial length of the neck N to provide stable securement to the bone. The lower surface 816 of component 802b is positioned on the surface C of the bone F from which the head portion has been removed. FIGS. 21A and 21B illustrate device 800 installed on a bone, in this instance a femur F. The same principles can apply to any other bone onto which there is a need for installing an implant according to the present disclosure.

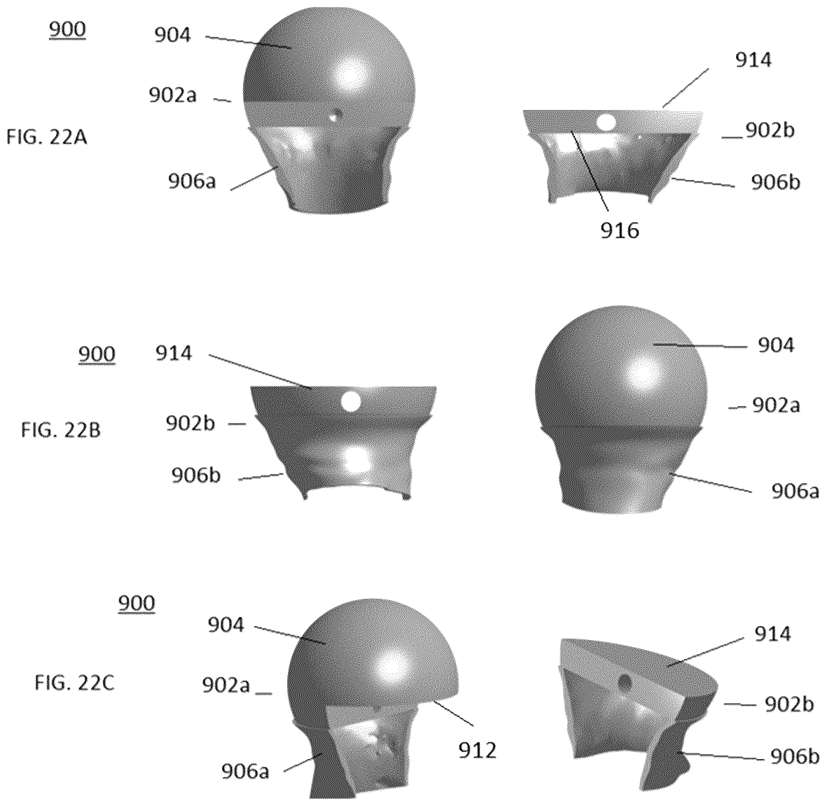

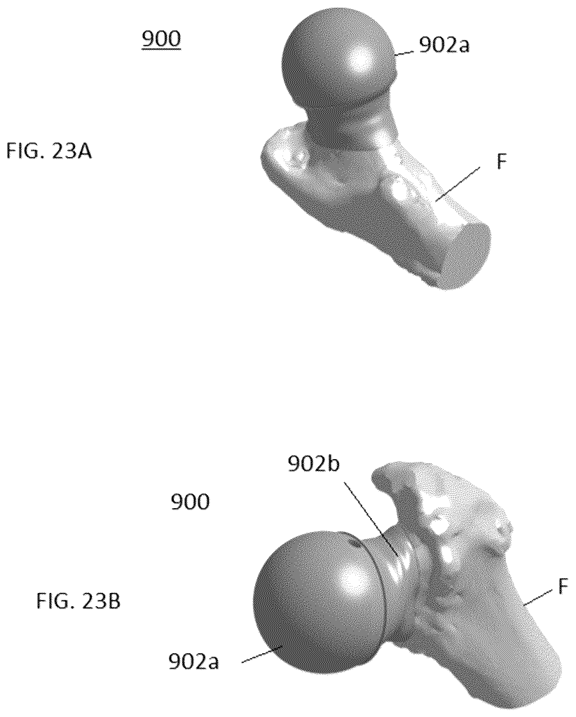

FIGS. 22A-22C illustrate an exemplary embodiment of device 900 that is also adapted for implantation onto a bone that participates in a ball-and-socket joint, such as at the hip or shoulder. Device 900 is substantially the same as device 400, illustrated above in FIGS. 7A-7D, with the differences noted here. Like device 700, shown in FIGS. 18A-18C, this device is cut along the horizontal plane anatomically producing superior and inferior halves. The cut is interrupted immediately above the proximal end of the neck to avoid a cut along the ball except near its base. The reduced seam on the ball presents a smoother surface for articulation in the socket. Device 900, like device 400, is asymmetric in some embodiments, in which a majority of the overlying ball 904 is part of one component 902a to minimize seam length in the bone socket, such as the hip or shoulder socket. The second component 902b interlocks using a horizontal fastening mechanism. As illustrated in FIGS. 22A-22C, component 902a includes ball 904 and collar 906a. Component 902b includes collar 906b. This neck portion 906a/b is configured to be longer in order to engage a substantial length of the neck N to provide stable securement to the bone. When component 902a is secured to component 902b, the lower surface 912 of ball 904 mates with the upper surface 914 of component 902b. The lower surface 916 of component 802b is positioned on the surface C of the bone F from which the head portion has been removed. FIGS. 23A and 23B illustrate device 900 installed on a bone, in this instance a femur F. The same principles can apply to any other bone onto which there is a need for installing an implant according to the present disclosure.

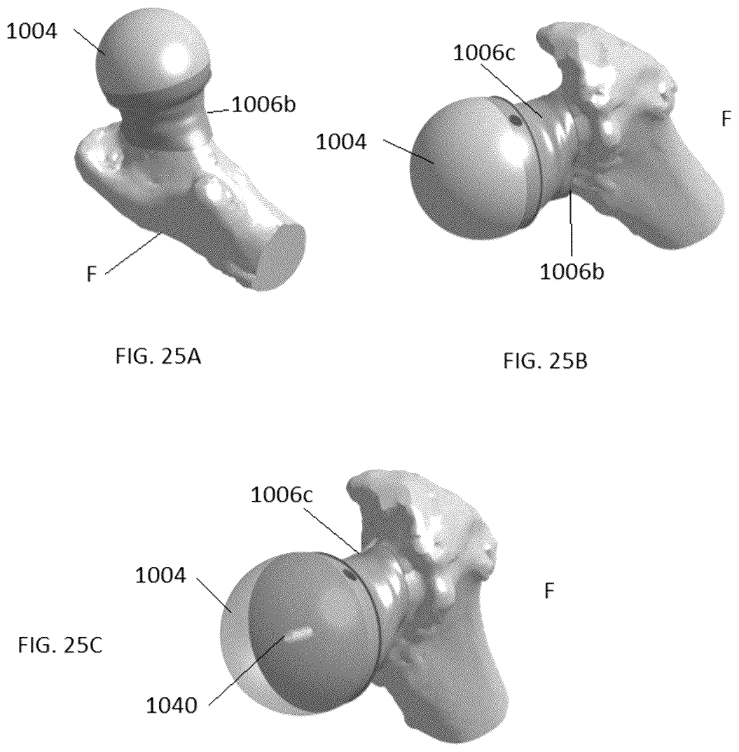

FIG. 24 illustrates an exemplary embodiment of device 1000. Device 1000 introduces a third prosthetic piece that has an attachment point on the proximal portion of the collar but as a separate piece can be manufactured from a different material than the collar. Device 1000, which in this instance is adapted for implantation onto a bone that participates in a ball-and-socket joint, such as at the hip or shoulder, includes three components 1002a, 1002b and 1002c, which are assembled about the joint portion of a bone F. Components 1002a includes the ball portion 1004. In other embodiments, a different implant head configuration can be used in place of ball portion 1004, such as a substantially planar head, a concave head, or any other configuration corresponding to the original shape of the now-truncated bone onto which the implant is to be secured, i.e., such as to replicate the original shape of the bone end that participates in the joint or to represent a reverse member of the joint (such as to represent the socket member of a ball-and-socket joint). Components 1002b and 1002c include the collar portions 1006a and 1006b respectively. Device 1000 is assembled on the bone F, in this instance a femur, is illustrated in FIGS. 25A-25C. Components 1002b and 1002c are positioned about the neck N, such that collar portions 1006a and 1006b provide mechanical securement. In some embodiments, components 1002b and 1002c are provided with a complementary projection 1030 on one component and a recess 1032 on the other component. Threaded apertures 1038 and 1039 are provided on each of the components 1002a and 1002b to receive a bolt to secure those components together. Component 1002a is then secured to components 1002b and 1002c by placing the surface 1012 of component 1002a on the combined surfaces 1014b/1014c of components 1002b/1002c and secured by a bolt 1040 passing though threaded apertures 1036 (in component 1002a) and aperture 1037 (in component 1002b). See also FIG. 25C in which ball 1004 is rendered as transparent to allow visualization of bolt 1040.

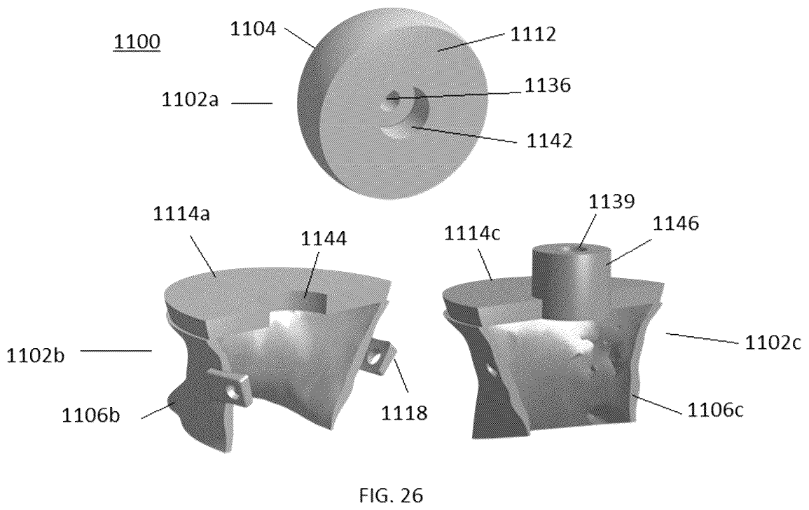

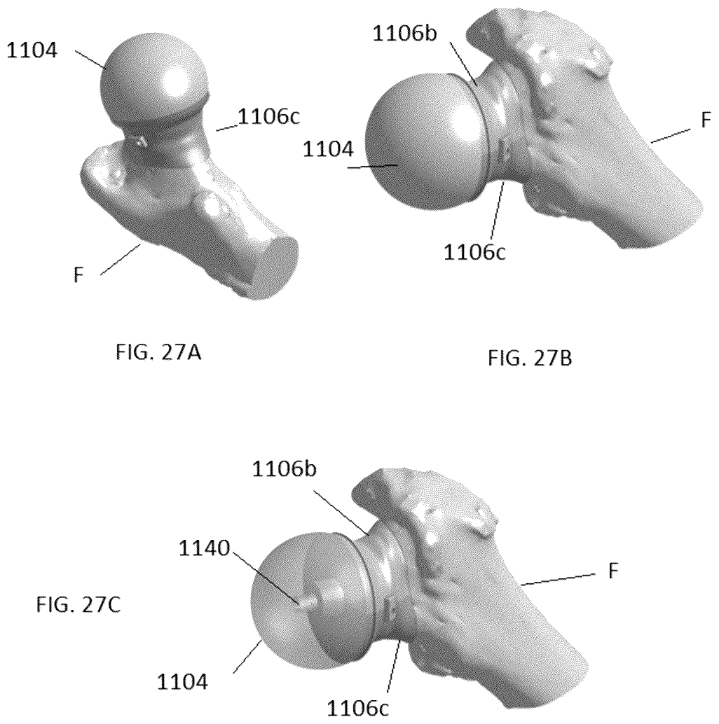

FIG. 26 illustrates an exemplary embodiment of device 1100, which in this instance is adapted for implantation onto a bone that participates in a ball-and-socket joint, such as at the hip or shoulder. Like device 1000 in FIGS. 24-25, the three-piece design allows for a separate head (here, a hemisphere or “ball”), and a larger fraction of the head is allowed by securing the collar pieces using external fastening points at the distal neck of the bone rather than internal fastening points under the ball in the proximal bone. This allows for a larger implant articulating surface. Device 1100 is substantially the same as device 1000 illustrated in FIG. 24, with the significant differences noted herein. Device 1100 includes three components 1102a, 1102b and 1102c, which are assembled about the hip joint portion of the femur F. Component 1102a includes the ball portion 1104. In other embodiments, a different implant head configuration can be used in place ball portion 1104, such as a substantially planar head, a concave head, or any other configuration corresponding to the original shape of the now-truncated bone onto which the implant is to be secured, i.e., such as to replicate the original shape of the bone end that participates in the joint. Components 1102b and 1102c include the collar portions 1106a and 1106b respectively. Device 1100 is assembled on the bone F, in this instance a femur, is illustrated in FIGS. 27A-27C. Components 1102b and 1102c are positioned about the neck N, such that collar portions 1106a and 1106b provide mechanical securement. In some embodiments, component 1102c is provided with a substantially longitudinally-extending cylindrical projection 1146 that is partially received in a complementary arcuate recess 1144 in component 1102b. Component 1102b and 1102c are provided with threaded recess, e.g., 1118 to receive a pair of screws or bolts to secure components 1102b and 1102c together. Component 1102a is then secured to components 1102b and 1102c by placing the surface 1112 of component 1102a on the combined surfaces 1114b/1114c of components 1102b/1102c. Component 1102a is configured with a cylindrical recess 1142 to receive the cylindrical projection 1146. Components 1102a/b/c are secured by a bolt 1140 passing though longitudinal threaded apertures 1136 (in component 1002a) and aperture 1139 (in component 1102b). See also FIG. 27C in which ball 1104 is rendered as transparent to allow visualization of bolt 1140.

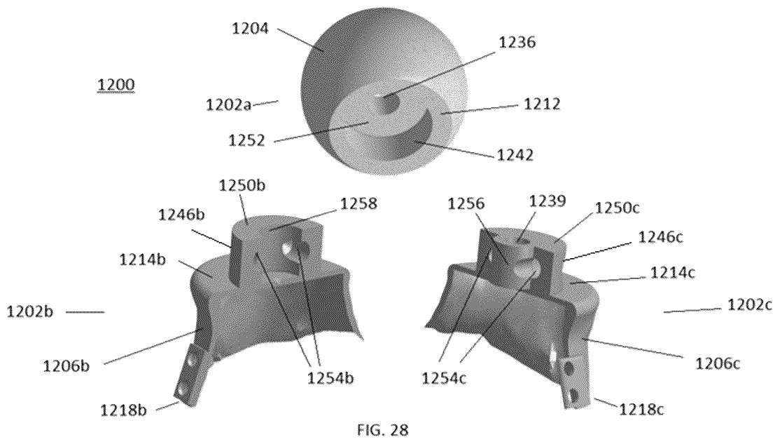

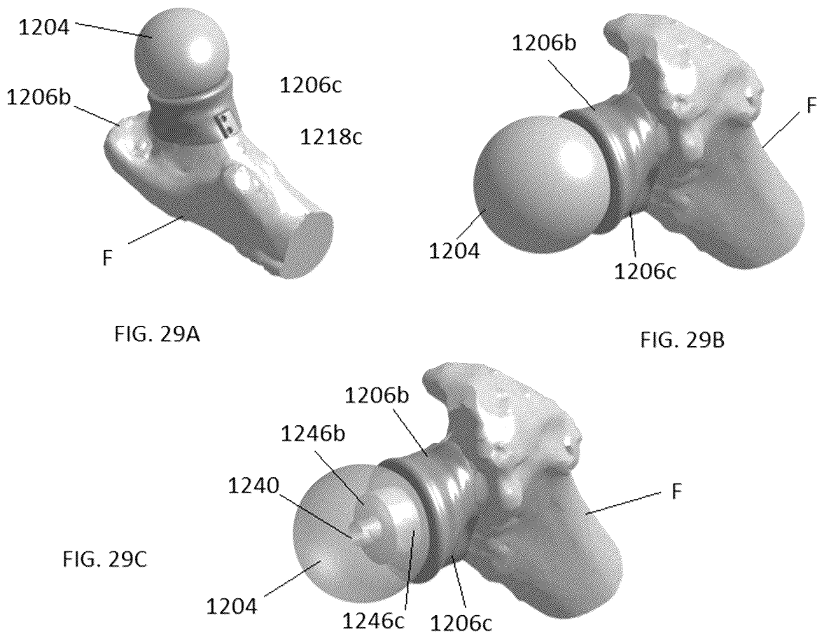

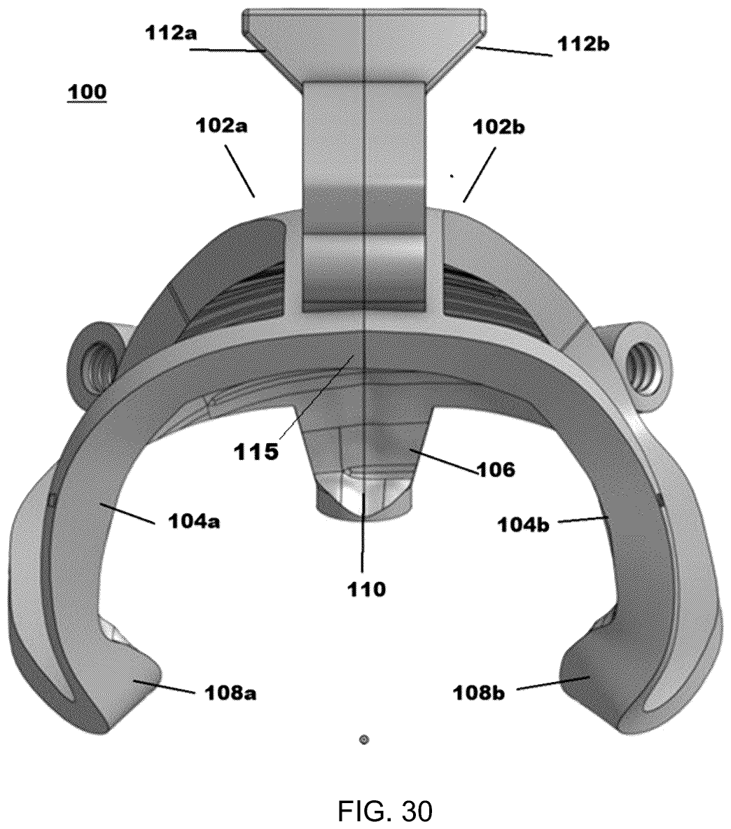

FIG. 28 illustrates an exemplary embodiment of device 1200, which in this instance is adapted for implantation onto a bone that participates in a ball-and-socket joint, such as at the hip or shoulder. In some embodiments, device 1200 includes a smaller head (here, hemisphere or “ball”) portion 1204, e.g., 36 mm diameter instead of a more typical 40 mm or greater. A smaller diameter ball would mate with a greater range of hip or shoulder socket liner options that have a smaller inner diameter than the native socket, and a smaller overall device diameter would limit impingement in the hip or shoulder socket during movement of the joint. Device 1200 is substantially the same as device 1100 illustrated in FIG. 26, with the significant differences noted herein. Device 1200 includes three components 1202a, 1202b and 1202c, which are assembled about the hip joint portion of the femur F. Component 1202a includes a ball portion 1204. In other embodiments, a different implant head configuration can be used in place ball portion 1204, such as a substantially planar head, a concave head, or any other configuration corresponding to the original shape of the now-truncated bone onto which the implant is to be secured, i.e., such as to replicate the original shape of the bone end that participates in the joint. Components 1202b and 1202c include the collar portions 1206a and 1206b respectively. Device 1200 is assembled on the bone F, in this instance a femur, is illustrated in FIGS. 29A-29C. Components 1202b and 1202c are positioned about the neck N, such that collar portions 1206a and 1206b provide mechanical securement. In some embodiments, components 1202b and 1202c are provided with a substantially longitudinally-extending cylindrical projection 1246b/1246c. A pair of transversely extending bores 1254b/1254c are provided in 1202b and 1202c, respectively, that are configured to received a pair of bolts to secure components 1202b and 1202c together. Component 1202c is also provided with a cylindrical projection 1256 that is partially received in a complementary arcuate recess 1258 in component 1202b. A first surface 1214b/1214c is defined at the top of collar portions 1206b/1206c. A second surface 1250b/1250c is defined at the top of cylindrical projection 1246b/1246c. Components 1202b and 1202c are provided with threaded recess, e.g., 1218b/1218c to receive a pair of screws or bolts to secure components 1102b and 1102c together.

Component 1202a includes surface 1212 and a cylindrical recess 1242 to receive the cylindrical projection 1246b/1246c defining an inset surface 1252. Component 1202a is then secured to components 1202b and 1202c by placing the surface 1212 of component 1202a on the combined surfaces 1214b/1214c, and the insert surface 1252 on the combined surfaces 1250b/1250c of components 1002b/1002c an. Components 1202a/b/c are secured by a bolt 1240 passing though longitudinal threaded apertures 1236 (in component 1202a) and aperture 1239 (in component 1202c). See also FIG. 29C in which head 1204 (here, a hemisphere or “ball”) is rendered as transparent to allow visualization of bolt 1240.

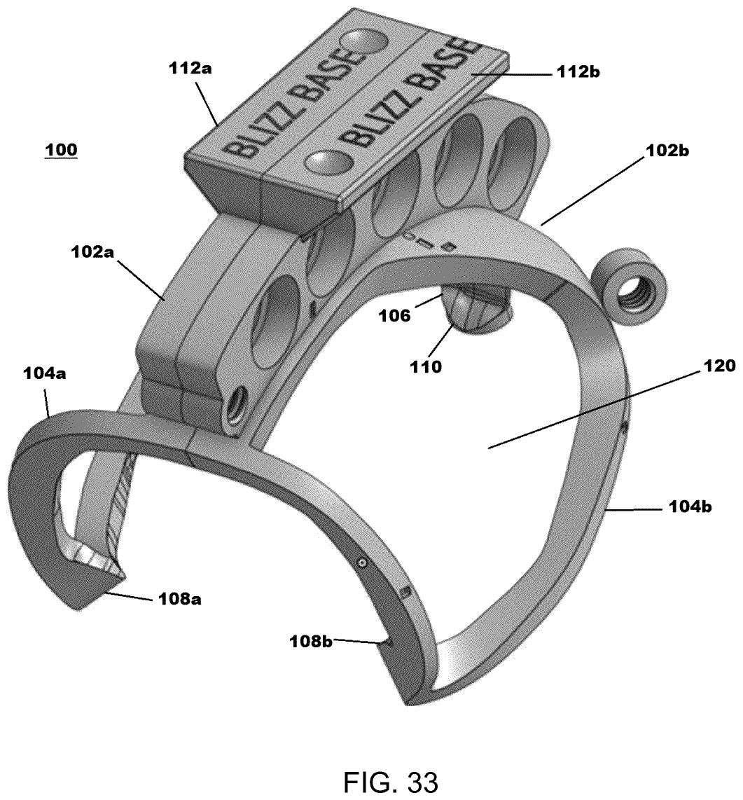

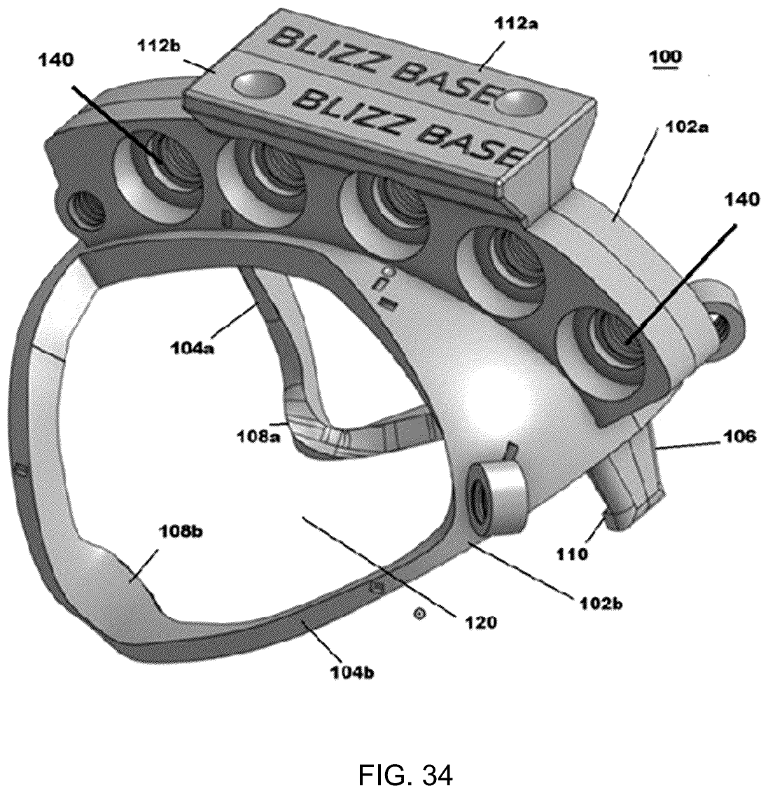

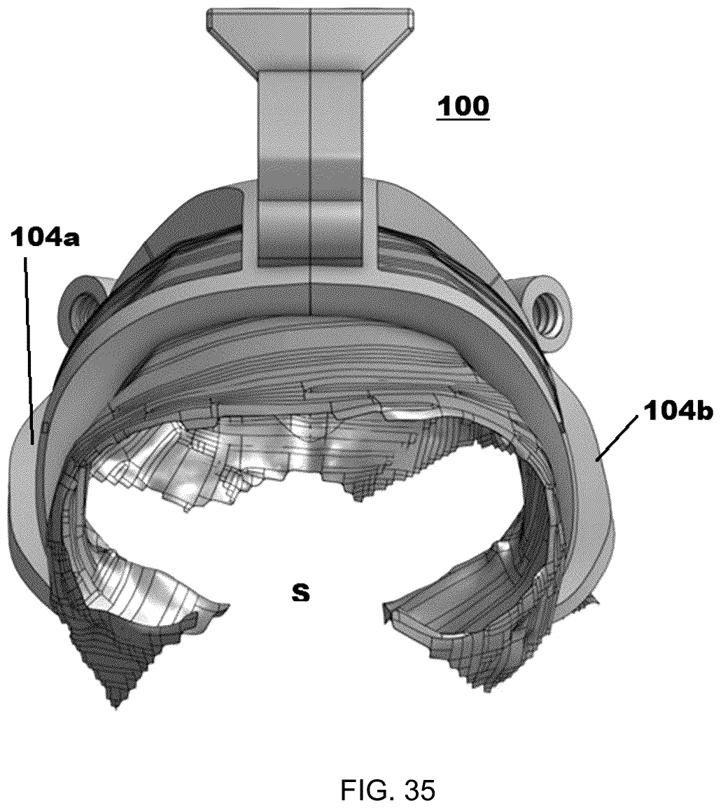

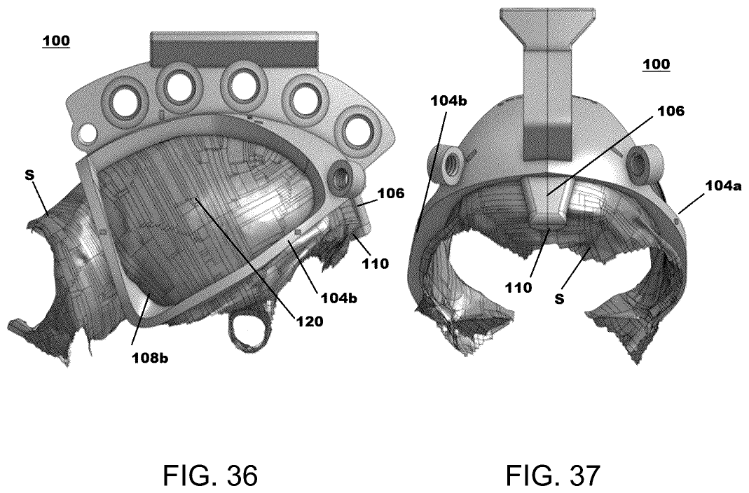

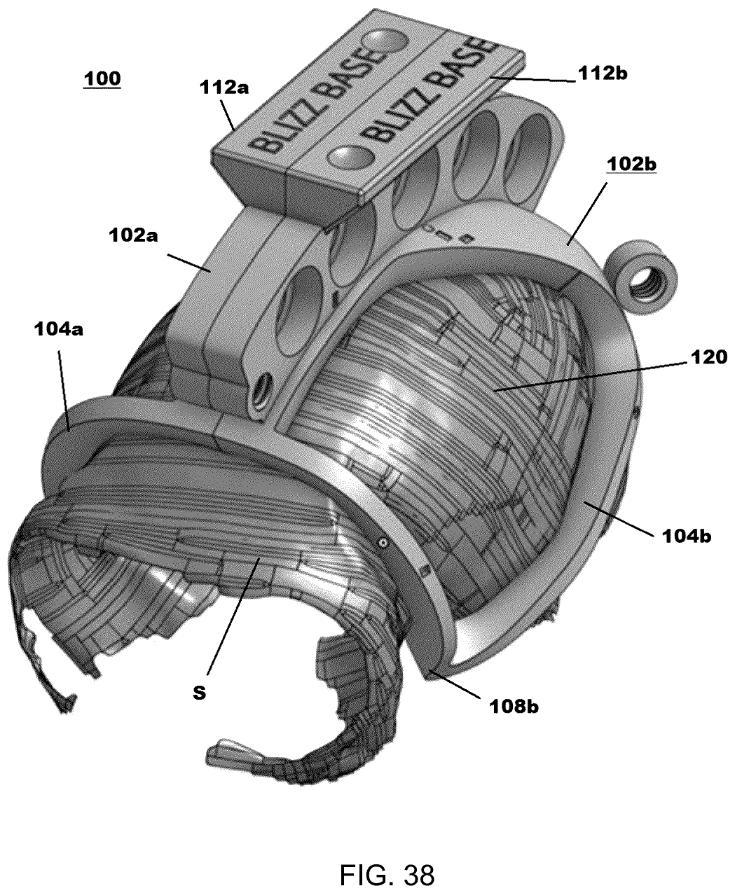

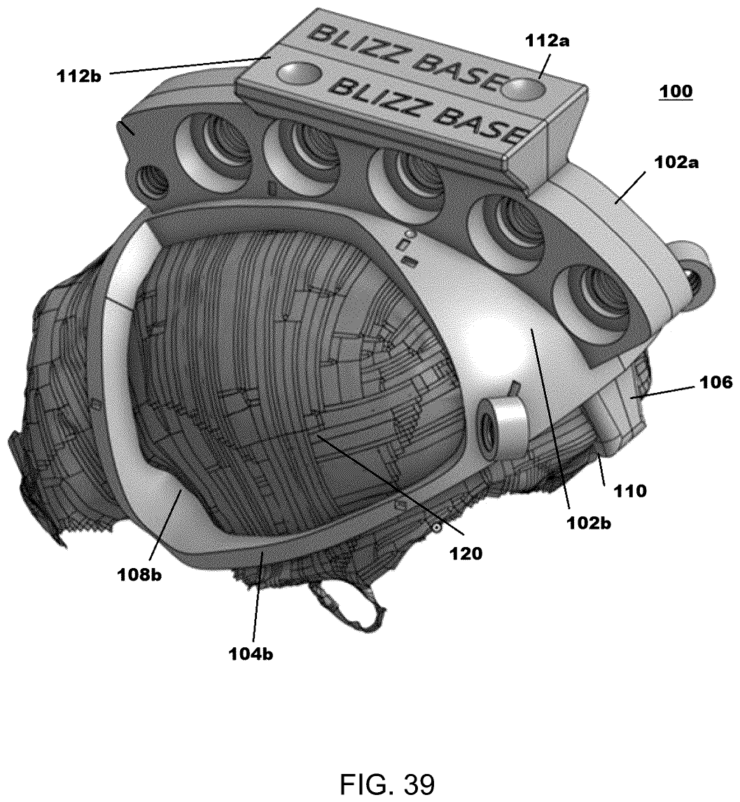

A further exemplary embodiment of a device for non-infiltrating mechanical securement to hard tissue, is implant 100 illustrated in FIGS. 30-34. Implant 100 is a screwless implant that requires no modification of the hard tissue, e.g., skull, and thus is relatively non-invasive compared to using screws that penetrate the skull or methods using foreign adhesives to bond to the skull surface.

In some embodiments, implant 100 uses the convex geometry of the skull itself to mechanically adhere a fitted implant, e.g., a titanium 3D-printed implant, to the skull. The novel device wraps around the curved geometry of the skull itself with no further modification of the skull, thus maintaining its integrity. In some embodiments, the device wraps around the underside of the skull in a tripod pattern at three key points: (1) occipital pole, (2) left and (3) right temporal poles. This geometry keeps the implant firmly fixed relative to the skull as it prevents lateral movements (arms of the tripod), vertical movement (by grabbing underhangs of skull poles), effectively limiting pitch/roll movements.

The extensive geometry distributes forces across the skull with the attendant benefit of allowing wide neural access. This “neural breadboard” allows mounting in a fixed/stable fashion any devices in the window as well as additional supporting hardware like signal processing circuits, wireless transmitter, and battery packs.

The implant can be made from a strong, biocompatible material. For example, titanium is a preferred material in an exemplary embodiment, and other viable options include natural PEEK or hard plastic. Titanium's biocompatibility fosters bone growth into the microgaps between the implant and the original skull, and this osseointegration in turn further integrates skull and implant as one.

Additionally, the novel implant is longer-lasting because its mechanical purchase is more fault-tolerant than typical implants. The mechanical purchase of the device is broad-based, driven by the purchase across the global skull geometry. Thus, the broad-based mechanical purchase of our implant design is longer-lasting than a screw-based approach, as infection and skull degradation are reduced in risk of occurrence as well as in their mechanical consequences if they do occur, effectively improving long-term acceptance and biocompatibility. Increasing the implant's longevity makes it generally safer, as a longer-lived device reduces the likelihood of future surgeries which are inherently risky

The design also allows a stronger implant, with greater mechanical stability. For high load applications, forces-whether rotation, sheer, lever arm, etc.—are distributed across the skull's surface, resilience to external forces and blunt trauma such as from falls.

Another benefit of this approach is that the device, essentially a helmet, can allow an expanded breadth of neural access. This kind of device would be of wide application to animal neurophysiology labs today that require mechanical rigidity of any mounted instrument to ensure precision and repeatability of experimental measurements. The device and technique could be applied as a scaffolding, a readily extendable lattice supporting the implementation of a variety of devices, such as electrical, chemical, magneto, and/or ultrasonic stimulation and recording-multimodal solutions will also be useful with such broad access—or the implant can be advantageous simply for wider brain access of a single modality. And for a minimally invasive neurophysiology approach in humans, it can be coupled with implanted, subepidermal electrodes below the scalp but above the skull, enabling subepidermal electroencephalography rather than the more invasive electrocorticography beneath the skull which involves placing electrodes on the surface of or even penetrating the brain's cortical sheet. Medical applications of such an approach are broad and include, for instance, the detection and localization of seizures for subsequent surgical removal.

Being precisely conformed to the skull surface, the implant is registered in stereotaxic, skull-referenced coordinates, so that it effectively provides an inline stereotax for targeting devices within the broad access window. The exact skull geometry for forming the interior surface of the implant is obtained using routine computed tomography (CT) scanning of the cranium at high resolution, but any imaging modality with contrast at the bone-soft tissue interface can suffice.

In some embodiments, implant for securement to the skull includes a first body portion having a first arm defining a first engagement portion for engagement with a first portion of the skull of the subject; a second body portion having a second arm defining a second engagement portion for engagement with a second portion of the skull of the subject; a rear arm extending from one or both of the first body portion and the second body portion, the rear arm defining a rear engagement portion for engagement with a third portion of the skull of the subject. A securement mechanism for coupling the first and second body portions about the skull of the subject is provided in some embodiments.

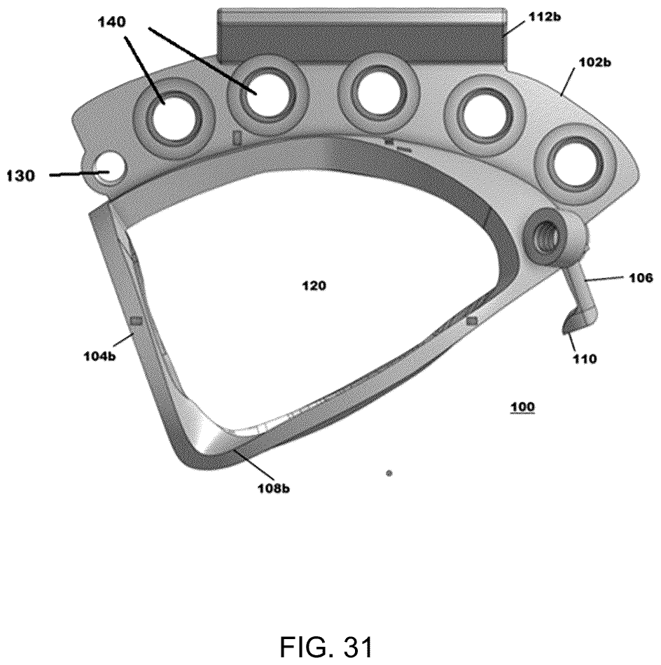

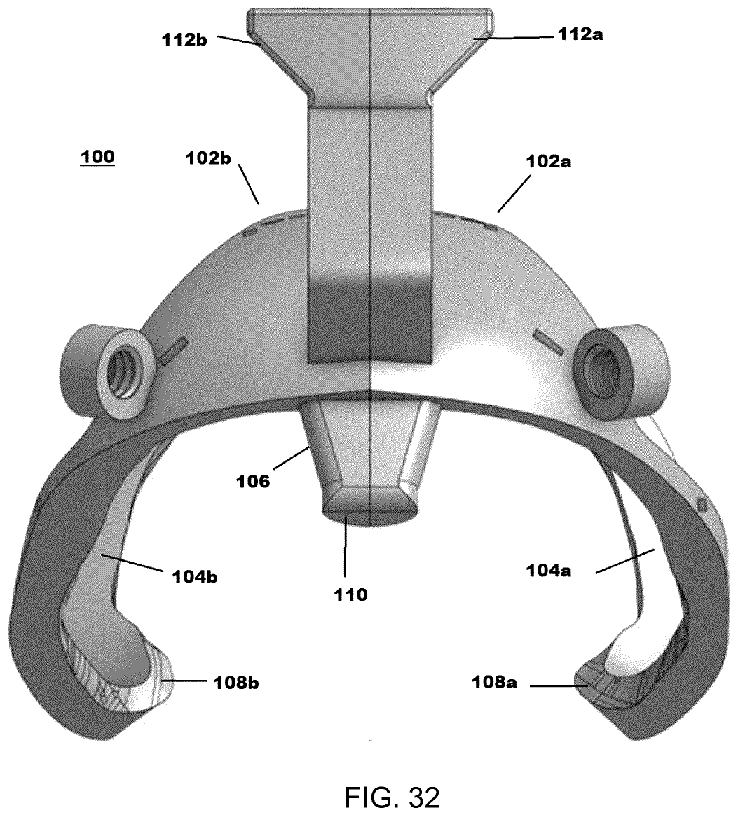

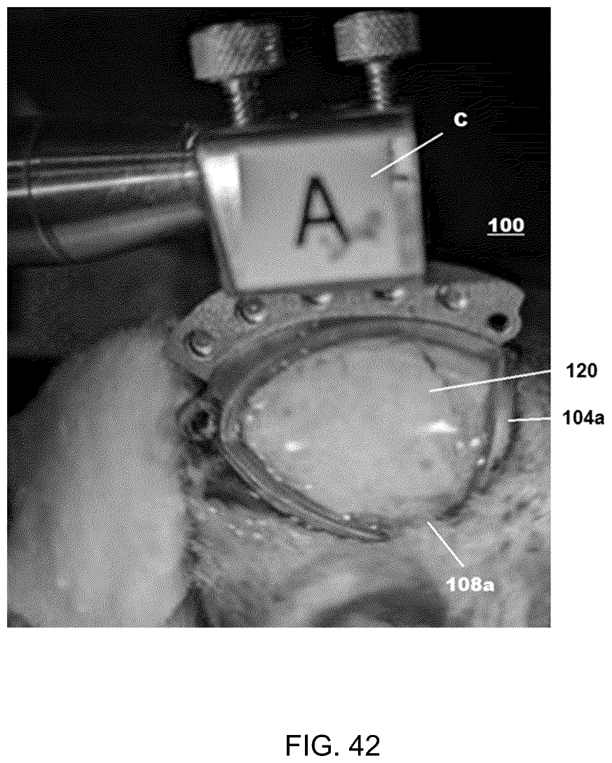

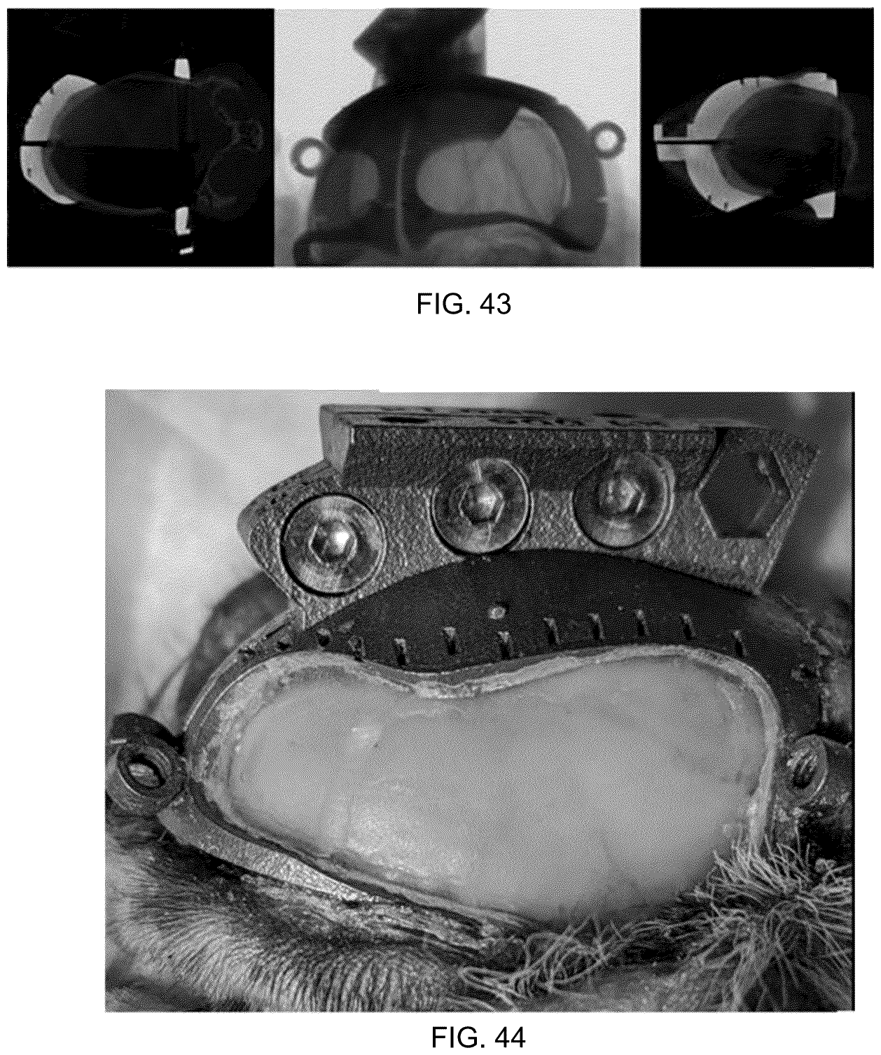

In some embodiments, implant 100 includes two body portions 102a/b that are substantially mirror images of one another about the medial plane. As discussed below, it is understood that body portion 102a and 102b can include shape differences to reflect asymmetrics in the skulls of the subjects. Each body portion 102a/b includes arm 104a/b that defines an opening or window 120. The posterior portion of the implant 100 is a rear arm 106. In some embodiments, the rear arm 106 includes two halves, each of which is formed on its respective body portion 102a/b. In some embodiments, the rear arm 106 is formed on one of the body portions 102a/b. Each arm 104a/b include a distal engagement portion 108a/b that is shaped and configured to engage a portion of the skull of the subject, as will be described in greater detail herein. Rear arm 106 include a rear engagement portion 110 that is shaped and configured to engage a portion of the skull of the subject. The left and right body portions 102a/b are designed to move closer together and further apart from the medial plane. A securement mechanism is provided to couple body portions 102a/b. In some embodiments, a plurality of screw holes 140 are provided along the top portion of each body portion 102a/b. One or more screws are provided in order to secure the body portions 102a/b together. For example, one body portion may include a series of openings, and the other body portion includes threaded openings to secure the body portions together via screws. Body portions may be coupled by clamps, adhesives in addition to or in alternative to screws. In some embodiments, a threaded aperture 130 (FIG. 31) can be used as mount points for attachments such as neural interfaces over the window or window cover when the window is not needed such as when experiments are being performed in the alternate hemisphere at a given time. Each body portion 102a/b includes a base portion 112a/b that is configured to be engaged by a clamp C to support the implant 100 in case of need to hold the head in a fixed position. (FIG. 42).

In some embodiments, the two body portions 102a/b are substantially symmetrical. In some embodiments, the body portions are asymmetrical (not shown). For example, the body portions can be asymmetrical about the midline. In some embodiments, there may be a plurality of body components that are assembled about the skull and secured without screws or adhesives, such as a snap fit.

Implant 100 is mechanically attached to hard tissue, in this case the skull. The shape of skull has a direction of curvature such that over at least one curve, a total curvature greater than 180 degrees is traversed if enough of the curves is traced out. For non-infiltrating mechanical securement, each arm 104a/b include a distal engagement portion 108a/b that extends far enough to reach around the lower edges of the curve (beyond 180 degrees) to engage the underhang, thereby preventing two degrees of axial motion in that plane.

The implant 100 has been implanted successfully in the common marmoset, Callithrix jacchus (n=6 animals). Implant 100 can be implanted in smaller mammals, e.g., mice, rats, and larger mammals, e.g., macaques and humans. Minor modifications are expected to accommodate the additional species, as all skulls have a generic closed, bounded geometry with curvature around occipital and temporal lobes, affording an underhang that the device wraps around to achieve mechanical purchase without bone screws or adhesive.

Materials that are biocompatible, strong, and that can be 3D printed at high resolution are preferred to manufacture the implant. Titanium meets all three criteria. Ceramic and natural PEEK can also be used but are less biocompatible, have less strength-to-weight ratio, and cannot be printed at high resolution in current commercially available printers. However, instead of 3D printing, ceramic and PEEK can be machined and are viable materials for our implanted device in a biological organism.

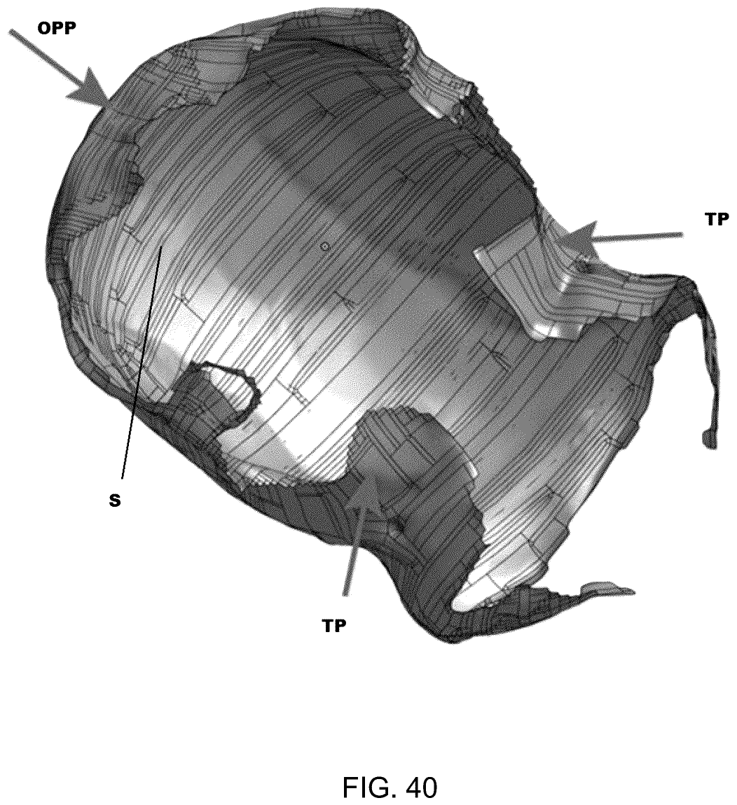

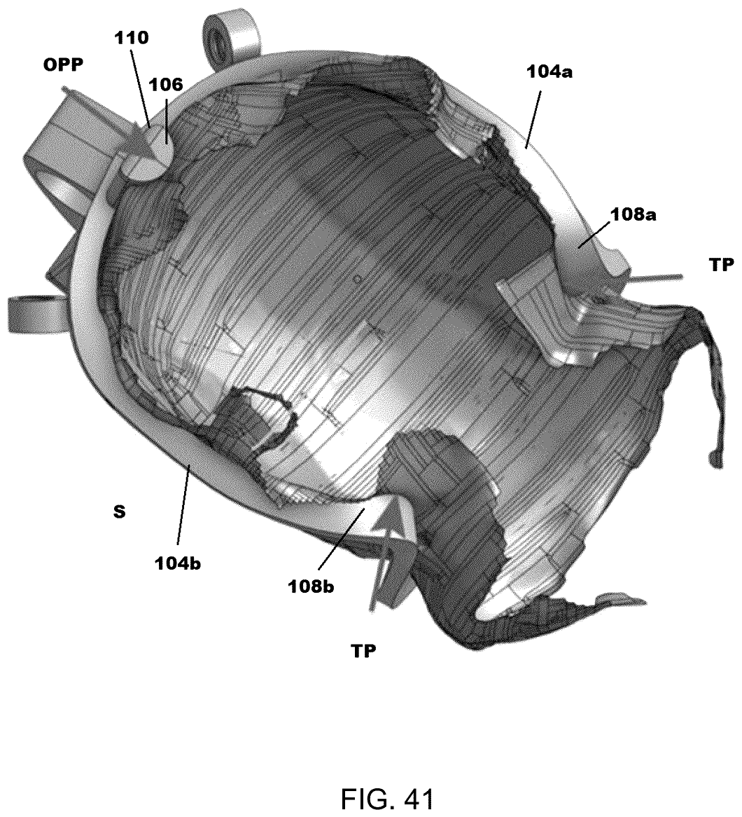

Design considerations for the implant are to take away degrees of freedom in all three translational dimensions (x,y,z) and both rotation angles (pitch & roll) for the implant not to move relative to the skull. As illustrated in FIGS. 40-41, three contact points on the skull S of the subject are shown. The contact point provide the form of a tripod geometry with vertices at the occipital pole protrusion OPP and left/right temporal poles TP (1) over the occipital ridge in the posterior (back center) of the skull and over the (2) right & (3) left temporal pole in the anterior, inferior skull. The tripod geometry prevents lateral motions. At the three contact points, the skull has a vertical convexity as it encases the curving brain. The implant grabs the lower surface of this curve to prevent up-down motion in the vertical plane. For rotational stability, the back vs. front contacts of the tripod prevent pitch, while the two lateralized front contacts prevent left-right roll.

Force closure to mechanically secure the implant to the skull is provided by four contact points on the skull. The implant 100 provide three contact points arranged such that they span greater than 180 degrees of total curvature of the end surface interface in the plane they define. In some embodiments, two contact points are provided by engagement surfaces 108a/b to engage the left/right temporal poles, and a third contact point 115 is provided along the interior surface of arms 104a/104b, such as at the center of the top of the skull. See, e.g., FIG. 30. The fourth contact point, which is not in the plane defined by the three contact points, is provided by a rear engagement portion 110 of rear arm 106 at the occipital pole protrusion. In some embodiments, instead of the occipital pole, the nuchal crests on the lateral skull could be used so that the anterior-posterior extent of the implant is smaller. Thus, the rear arm can engage any number of points along the medial-to-lateral extent of occipital bone ridge near where the cervical (neck) muscles insert (e.g., the superior nuchal line as well as inferior nuchal line), extending from the occipital prominence at the midline in the posterior skull to the nuchal crests more laterally and anteriorly. It is understood that the structure of the implant, including the arms 104a/b and 106 and engagement portions 108a/108b/110 may be provide additional contact points.

The implant 100 is designed in two halves, body portion 102a/b, so that it can be installed by clamping across the midline (halves connected by screws at the midline, not shown). Fastening screws can be oriented either tangentially or orthogonally to the implant surface to minimize the vertical profile of the implant center. Instead of screws, various bonding agents and glues could be used for fastening implant pieces. However, securement by bonding agents is less reversible for uninstalling. Snap fits could be used and may be made reversible if the tabs have some flexibility. Snap fits provide a way to lower the vertical profile of the implant over the skull surface, making it easier to cover with the scalp.

In some embodiments, the implant could be divided into three separate pieces corresponding to the three legs of the tripod (arms 104a/b, 110) that would then be fastened together at the midline with screws or bonding agents. Three pieces might be easier to slide into position under the overlying temporalis muscle and scalp soft tissue. Compared to the three straps/leg pieces which have a minimalist planar geometry for installation, clamping two large geometrically complex pieces as in implant 100 potentially requires a wider surgical dissection to position the two pieces onto the skull. Furthermore, the intersection of the three arms in the tripod can be moved further back or forward along the midline depending on where midline neural access is desired. However, the two-piece design allows building in large windows for neural recording in each hemisphere or mounting devices within a window.

The implant is installed by first resecting the scalp and the underlying fascia to expose the temporalis muscle underneath. Two approaches can be used with regard to the large temporalis muscle overlying the skull including access to the implant's engagement points in the temporal poles of the skull. In one approach, the temporalis muscle is retracted and severed at its base near the zygomatic arch. This approach then leaves the skull over the cerebral cortex completely accessible to windows in the implant which can be useful for experimental settings in animals. In practice, other muscles compensate for the lost temporalis muscle such that animals retain mouth movements for chewing and vocalizing. In human patient settings where invasiveness of the procedure and cosmetic outcomes are overriding factors, a second approach for implant installation is to slide the implant under the temporalis but retain the muscle (or do a partial removal). This can be done either by cutting a slit in the tendon at its insertion into the temporal bone ridge whose width accommodates sliding in of the implant leg or by resecting the muscle entirely at various points of insertion and then re-attaching over the implant leg once installed.

During installation to allow for more precise fit of the implant, multiple scales can be tested. This is done to accommodate the possibility of small errors either in skull estimation or in the implant manufacturing process since these have limited resolution. Slightly scaled down versions of the same implant can provide a tighter fit and slightly scaled up versions overcome any tolerance issues if skull geometry is not perfectly estimated creating interference points.

The implant is removable by blunt dissecting any overlying soft tissue that has adhered over top such as muscle or fascia, by then unscrewing at the fastening point of the pieces at the top of the skull, and then just sliding the pieces off the skull surface.

Monitoring devices can be used through the windows 120 of the implant 100. For example, (a) optical microscopy methods which can sense light from the brain or vasculature, through the skull or directly at the brain surface; (b) ultrasound recording or stimulation methods which transduce or receive soundwaves through the skull; or (c) multielectrode arrays for high temporal precision recordings of brain electrical activity can be used.

Implant 100 can serve as a “neural breadboard.” 3D printing makes it possible to configure arbitrary scaffolds for mounting devices within the window 120. Any configuration/weight device can be accommodated-including attendant hardware for signal processing, signal transmission (e.g., wireless communication), and battery supply-because of the strength of using a broad surface-to-surface contact that distributes forces while providing a breadth of mounting points. The stereotaxically placed marks illustrate the inline skull-referenced coordinate system provided by the implant for mounting and targeting device.

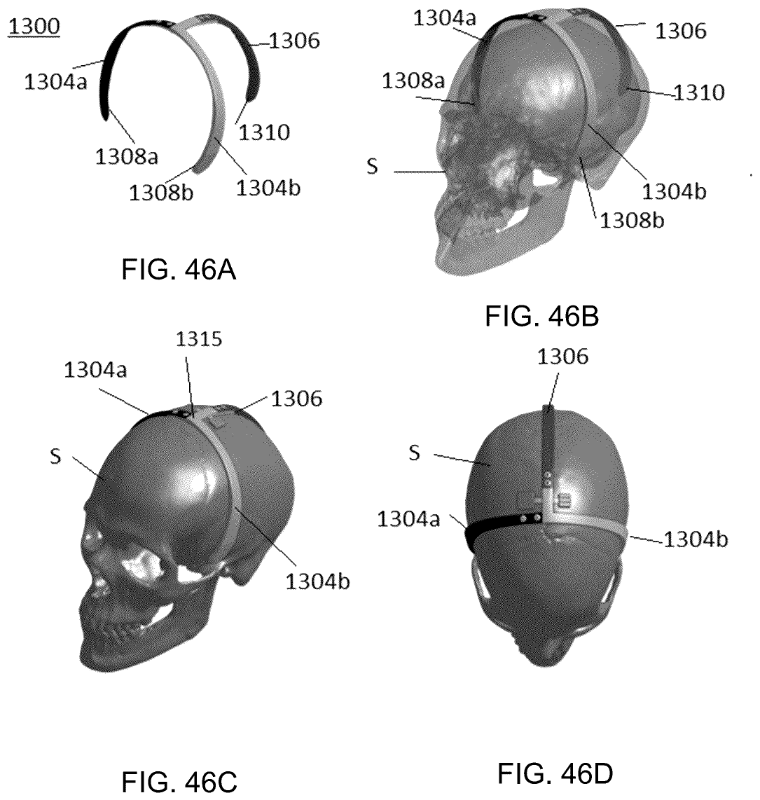

A further exemplary embodiment of an implant 1300 is illustrated in FIGS. 45A-C and FIGS. 46A-D for implantation on the skull of a primate or human skull S. In some embodiments, implant 1300 is a scaffold for securing a battery pack and stimulator, and includes a pair of side arms 1304a/1304b and a rear arm 1306. In some embodiments, the rear arm 1306 is located at the posterior portion of the implant 1300. Each arm 1304a/b include a distal engagement portion 1308a/b that is shaped and configured to engage a portion of the skull of the subject, as will be described in greater detail herein. Rear arm 1306 includes a rear engagement portion 1310 that is shaped and configured to engage a portion of the skull S of the subject. In some embodiments, a plurality of screw holes are provided along the top portion of each body portion 102a/b. One or more screws are provided in order to secure the body portions 102a/b together. For example, one body portion may include a series of openings, and the other body portion includes threaded openings to secure the body portions together via screws. Body portions may be coupled by clamps, adhesives in addition to or in alternative to screws.

Force closure to mechanically secure the implant 1300 to the skull is provided by four contact points on the skull. The implant 1300 provide three contact points arranged such that they span greater than 180 degrees of total curvature of the end surface interface in the plane they define. In some embodiments, two contact points are provided along the length of arms 1304a/b, e.g., at engagement portions 1308a/b and a third contact point 1315 is provided along the interior surface of arms 1304a/1304b, such as at the center of the top of the skull. See, e.g., FIG. 46C. The fourth contact point, which is not in the plane defined by the three contact points, is provided by a rear engagement portion 1310 of rear arm 1306, e.g., at the occipital pole protrusion. It is understood that the structure of the implant, including the arms 1304a/b and 1306 and engagement portions 1308a/1308b/1310 may be provide additional contact points.

In some embodiments, the two side arms 1304a/1304b are substantially symmetrical. In some embodiments, the body portions are asymmetrical (not shown). For example, the body portions can be asymmetrical about the midline. In some embodiments, there may be a plurality of body components that are assembled about the skull and secured without screws or adhesives.

As discussed above regarding implant 100, implant 1300 is mechanically attached to hard tissue, in this case the skull. The shape of skull has a direction of curvature such that over at least one such portion, a total absolute curvature greater than 180 degrees is traversed if enough of the curves is traced out. For non-infiltrating mechanical securement, each arm 1304a/b and 1306 include a distal engagement portion 1308a/b and 1310 that extends far enough to reach around the lower edges of the curve (beyond 180 degrees) to engage the underhang, thereby preventing two degrees of axial motion in that plane.

In some embodiments, implant 100 and/or implant 1300 further includes a solid shield, cover or sheath that extends between the side arms and the rear arm to cover the skull. In some embodiments, the shield may contact the skull and in some embodiments, it is spaced apart from the skull.

In some embodiments, devices 200, 300, 400, 500, 600, 700, 800, 900, 1000, 1100, and 120 described herein may secure to the neck of a bone, such as the femur, humerus, radius, or ulna by three distinct contact points.

While the disclosed subject matter is described herein in terms of certain non-limiting exemplary embodiments, those skilled in the art will recognize that various modifications and improvements may be made to the disclosed subject matter without departing from the scope thereof. Moreover, although individual features of one embodiment of the disclosed subject matter may be discussed herein or shown in the drawings of the one embodiment and not in other embodiments, it should be apparent that individual features of one embodiment may be combined with one or more features of another embodiment or features from a plurality of embodiments. In addition to the specific embodiments claimed below, the disclosed subject matter is also directed to other embodiments having any other possible combination of the dependent features claimed below and those disclosed above. As such, the particular features presented in the dependent claims and disclosed above can be combined with each other in other manners within the scope of the disclosed subject matter such that the disclosed subject matter should be recognized as also specifically directed to other embodiments having any other possible combinations. Thus, the foregoing description of non-limiting example embodiments of the disclosed subject matter has been presented for purposes of illustration and description. It is not intended to be exhaustive or to limit the disclosed subject matter to those embodiments disclosed herein.

Aspects. The following Aspects are illustrative only and do not limit the scope of the present disclosure or the appended claims. Any part or parts of any one or more Aspects can be combined with any part or parts of any one or more other Aspects.

Aspect 1. A prosthesis for securement to an end portion of a bone representing a first member of a joint in a subject comprising:
a cap portion for that is configured for fitting over the end portion of the bone; and
a collar configured for coupling to the cap portion and comprising a first collar portion and a second collar portion, at least one of said first collar portion and said second collar portion being coupled to the cap portion, wherein an interior surface of the collar conforms to a portion of an outer surface of a side portion of the bone extending from the end portion; wherein the first collar portion is configured to be coupled to the second collar portion to at least partially engage the outer surface of the side portion of the bone to provide mechanical securement of the collar with the bone.

Aspect 2. The prosthesis of aspect 1, wherein the side portion of the bone has an end surface defining a direction of curvature such that over at least a portion of the surface, a total curvature of at least 180 degrees is defined, the collar comprising at least four contact points, wherein three contact points of the four contact points span over 180 degrees of total curvature of the side portion in a plane defined by the three contact points, and a fourth contact point outside the plane, the four contact points providing mechanical securement of the body portion to the hard tissue.

Aspect 3. The prosthesis of aspect 1, further comprising a bolt to secure the first collar portion and the second collar portion.

Aspect 4. The prosthesis of aspect 1, wherein the first collar portion is symmetric with the second collar portion.

Aspect 5. The prosthesis according to aspect 1, wherein the prosthesis is for securement to the neck of a femur or humerus, whereby the cap portion comprises a ball portion for replacement of the femoral or humeral head, an interior surface of the collar conforms to a portion of the outer surface of the neck of the femur or humerus, and the first collar portion is configured to be coupled to the second collar portion in order to at least partially engage the outer surface of the neck of the femur or humerus to provide mechanical securement of the collar with the femur or humerus.

Aspect 6. The prosthesis of aspect 5, wherein the first collar portion and the second collar portion overlap the line of attachment of border of synovial membrane.

Aspect 7. The prosthesis of aspect 5, wherein the first collar portion and the second collar portion extends to the line of reflection of the synovial membrane.

Aspect 8. The prosthesis of aspect 1, wherein the cap portion is integral with the first collar portion.

Aspect 9. The prosthesis of aspect 1, wherein the cap portion comprises a first and second cap components, and wherein the first cap component is integral with the first collar portion and the second cap component is integral with the second collar portion.

Aspect 10. The prosthesis according to aspect 1, wherein the cap portion comprises at least a first cap element and a second cap element that are respectively coupled together during assembly of the prosthesis for securement to the end portion of the bone.

Aspect 11. The prosthesis according to aspect 10, wherein the first cap element and the second cap element respectively represent substantially equal halves of said cap portion.

Aspect 12. The prosthesis according to aspect 10, wherein the first cap element and the second cap element respectively represent unequally sized parts of said cap portion.

Aspect 13. The prosthesis according to aspect 12, wherein the first cap element includes a flange that fits over at least a portion of the second cap element during assembly of the prosthesis.

Aspect 14. The prosthesis according to aspect 10, wherein the cap portion includes a third cap element that fits over at least a portion of one or both of the first cap element and the second cap element during assembly of the prosthesis.

Aspect 15. An implant for securement to the end surface of hard tissue of a subject comprising:
a first arm;
a second arm; and
a third arm extending from one or both of the first arm and the second arm, the first arm, the second arm and the third arm comprising at least four contact points, wherein three contact points of the four contact points span over 180 degrees of total curvature of the end surface in a plane defined by the three contact points, and a fourth contact point outside the plane, the four contact points providing mechanical securement of the body portion to the hard tissue.

Aspect 16. The implant of aspect 15, further comprising a first body portion and a second body portion, and wherein the first arm extends from the first body portion and the second arm extends from the second body portion, and further comprising a securement mechanism for coupling the first and second body portions about the skull of the subject.

Aspect 17. The implant of aspect 15, wherein the hard tissue is the skull of the subject.

Aspect 18. The implant of aspect 17, wherein the first contact point is configured for engagement with the left temporal pole of the skull of the subject.

Aspect 19. The implant of aspect 17, wherein the second contact point is configured for engagement with the right temporal pole of the skull of the subject.

Aspect 20. The implant of aspect 17, wherein the first contact point and second contact point are configured for engagement with the nuchal crests of the skull of the subject.

Aspect 21. The implant of aspect 17, wherein the fourth contact point is configured for engagement with the occipital pole protrusion of the skull of the subject.

Aspect 22. The implant of aspect 15, wherein the implant is fabricated from titanium.

Aspect 23. The implant of aspect 15, wherein the subject is a marmoset monkey.

Aspect 24. The implant of aspect 15, wherein the first arm and the second arm define an access window extending therethrough.

Aspect 25. A device for mechanical securement to hard tissue of a subject, the hard tissue having an end surface defining a direction of curvature such that over at least one portion of the surface, a total curvature of at least 180 degrees is defined, the device comprising
a body portion comprising at least four contact points, three contact points of the four contact point span over 180 degrees of total curvature of the end surface in a plane defined by the three contact points, and a fourth contact point outside the plane, the four contact points providing mechanical securement of the body portion to the hard tissue.

Aspect 26. The device of aspect 25, wherein the hard tissue is bone or teeth.

Aspect 27. The device of aspect 25, wherein the hard tissue is the femur of a subject.

Aspect 28. The device of aspect 27, wherein the device is a hip joint prosthesis, wherein the body portion is a substantially spherical ball portion for replacement of the femoral head; and a collar coupled to the ball portion and comprising a first collar portion and a second collar portion, wherein the one or more engagement surfaces comprise an interior surface of the collar conforming to a portion of the outer surface of the neck of the femur; and wherein the first collar portion is configured to be coupled to the second collar portion to provide the at least four contact points.

Aspect 29. The device of aspect 28, further comprising a bolt to secure the first collar portion and the second collar portion.

Aspect 30. The device of aspect 28, wherein the first collar portion is symmetric with the second collar portion.

Aspect 31. The device of aspect 28, wherein the first collar portion and the second collar portion overlap the line of attachment of border of synovial membrane.

Aspect 32. The device of aspect 28, wherein the first collar portion and the second collar portion extends to the line of reflection of the synovial membrane.

Aspect 33. The device of aspect 28, wherein the ball portion is integral with the first collar portion.

Aspect 34. The device of aspect 28, wherein the ball portion comprises a first and second substantially hemispherical components, and wherein the first substantially hemispherical component is integral with the first collar portion and the second substantially hemispherical component is integral with the second collar portion.

Aspect 35. The device of aspect 28, wherein the ball portion is removably coupleable with the first collar portion and the second collar portion.

Aspect 36. The device of aspect 25, wherein the hard tissue is the skull of a subject.

Aspect 37. The device of aspect 36, comprising:
a first arm;
a second arm; and
a third arm extending from one or both of the first arm and the second arm, the first arm, the second arm and the third arm comprising at least four contact points, wherein three contact points of the four contact points span over 180 degrees of total curvature of the end surface in a plane defined by the three contact points, and a fourth contact point outside the plane, the four contact points providing mechanical securement of the body portion to the skull.

Aspect 38. The device of aspect 37, further comprising a securement mechanism for coupling the first, second and third arms about the skull of the subject.

Aspect 39. The device of aspect 38, wherein the first contact point is configured for engagement with the left temporal pole of the skull of the subject.

Aspect 40. The device of aspect 38, wherein the second contact point is configured for engagement with the right temporal pole of the skull of the subject.

Aspect 41. The device of aspect 38, wherein the first and second contact points are configured for engagement with the nuchal crests of the skull of the subject.

Aspect 42. The device of aspect 38, wherein the rear contact point is configured for engagement with the occipital pole protrusion of the skull of the subject.

Aspect 43. The device of aspect 38, wherein the implant is fabricated from titanium.

Aspect 44. The device of aspect 38, wherein the subject is a marmoset monkey, macaque, mouse or human.

Aspect 45. The device of aspect 38, wherein the securement mechanism comprises a screw inserted in threaded holes in the first and second arms.

Aspect 46. The device of aspect 38, wherein the first arm and the second arm define an access window extending therethrough.

1. A prosthesis for securement to an end portion of a bone representing a first member of a joint in a subject, comprising:
a cap portion that is configured for fitting over the end portion of the bone; and
a collar configured for coupling to the cap portion and comprising a first collar portion and a second collar portion, at least one of said first collar portion and said second collar portion being coupled to the cap portion, wherein an interior surface of the collar conforms to a portion of an outer surface of a side portion of the bone extending from the end portion; wherein the first collar portion is configured to be coupled to the second collar portion to at least partially engage the outer surface of the side portion of the bone to provide mechanical securement of the collar with the bone.

2. The prosthesis of claim 1, wherein the side portion of the bone has an end surface defining a direction of curvature such that over at least a portion of the surface, a total curvature of at least 180 degrees is defined, the collar comprising at least four contact points, wherein three contact points of the four contact points span over 180 degrees of total curvature of the side portion in a plane defined by the three contact points, and a fourth contact point outside the plane, the four contact points providing mechanical securement of the body portion to the hard tissue.

3. The prosthesis of claim 1, further comprising a bolt to secure the first collar portion and the second collar portion.

4. The prosthesis of claim 1, wherein the first collar portion is symmetric with the second collar portion.

5. The prosthesis according to claim 1, wherein the prosthesis is for securement to the neck of a femur or humerus, whereby the cap portion comprises a ball portion for replacement of the femoral or humeral head, an interior surface of the collar conforms to a portion of the outer surface of the neck of the femur or humerus, and the first collar portion is configured to be coupled to the second collar portion in order to at least partially engage the outer surface of the neck of the femur or humerus to provide mechanical securement of the collar with the femur or humerus.

6. (canceled)

7. (canceled)

8. The prosthesis of claim 1, wherein the cap portion is integral with the first collar portion.

9. The prosthesis of claim 1, wherein the cap portion comprises a first and second cap components, and wherein the first cap component is integral with the first collar portion and the second cap component is integral with the second collar portion.

10. The prosthesis according to claim 1, wherein the cap portion comprises at least a first cap element and a second cap element that are respectively coupled together during assembly of the prosthesis for securement to the end portion of the bone.

11. The prosthesis according to claim 10, wherein the first cap element and the second cap element respectively represent substantially equal halves of said cap portion.

12. The prosthesis according to claim 10, wherein the first cap element and the second cap element respectively represent unequally sized parts of said cap portion.

13. The prosthesis according to claim 12, wherein the first cap element includes a flange that fits over at least a portion of the second cap element during assembly of the prosthesis.

14. The prosthesis according to claim 10, wherein the cap portion includes a third cap element that fits over at least a portion of one or both of the first cap element and the second cap element during assembly of the prosthesis.

15. An implant for securement to the end surface of hard tissue of a subject comprising:
a first arm;
a second arm; and
a third arm extending from one or both of the first arm and the second arm, the first arm, the second arm and the third arm comprising at least four contact points, wherein three contact points of the four contact points span over 180 degrees of total curvature of the end surface in a plane defined by the three contact points, and a fourth contact point outside the plane, the four contact points providing mechanical securement of the body portion to the hard tissue.

16. The implant of claim 15, further comprising a first body portion and a second body portion, and wherein the first arm extends from the first body portion and the second arm extends from the second body portion, and further comprising a securement mechanism for coupling the first and second body portions about the skull of the subject.

17. (canceled)

18. (canceled)

19. (canceled)

20. (canceled)

21. (canceled)

22. (canceled)

23. (canceled)

24. The implant of claim 15, wherein the first arm and the second arm define an access window extending therethrough.

25. A device for mechanical securement to hard tissue of a subject, the hard tissue having an end surface defining a direction of curvature such that over at least one portion of the surface, a total curvature of at least 180 degrees is defined, the device comprising
a body portion comprising at least four contact points, three contact points of the four contact point span over 180 degrees of total curvature of the end surface in a plane defined by the three contact points, and a fourth contact point outside the plane, the four contact points providing mechanical securement of the body portion to the hard tissue.

26. (canceled)

27. (canceled)

28. The device of claim 25, wherein the device is a hip joint prosthesis, wherein the body portion is a substantially spherical ball portion for replacement of the femoral head; and a collar coupled to the ball portion and comprising a first collar portion and a second collar portion, wherein the one or more engagement surfaces comprise an interior surface of the collar conforming to a portion of the outer surface of the neck of the femur; and
wherein the first collar portion is configured to be coupled to the second collar portion to provide the at least four contact points.

29. (canceled)

30. The device of claim 28, wherein the first collar portion is symmetric with the second collar portion.

31. (canceled)

32. (canceled)

33. The device of claim 28, wherein the ball portion is integral with the first collar portion.

34. (canceled)

35. (canceled)

36. (canceled)

37. The device of claim 25, comprising:
a first arm;
a second arm; and
a third arm extending from one or both of the first arm and the second arm, the first arm, the second arm and the third arm comprising at least four contact points, wherein three contact points of the four contact points span over 180 degrees of total curvature of the end surface in a plane defined by the three contact points, and a fourth contact point outside the plane, the four contact points providing mechanical securement of the body portion to the skull.

38. (canceled)

39. (canceled)

40. (canceled)

41. (canceled)

42. (canceled)

43. (canceled)

44. (canceled)

45. (canceled)

46. (canceled)
